(19) United States
(12) Patent Application Publication
(10) Pub. No.: US 2003/0167531 A1
(43) Pub. Date: Sep. 4, 2003

(54) Expression and purification of bioactive, authentic polypeptides from plants

(76) Inventors: Douglas A. Russell, Madison, WI (US); Michael Schlittler, Wildwood, MO (US)

Correspondence Address: ARNOLD & PORTER, Attn: IP Docketing Department Room 1126B, 555 - 12th Street NW, Washington, DC, 20004-1206, US

(21) Appl. No.: 09/824,200

(22) Filed: Apr. 3, 2001

Related U.S. Application Data
(63) Continuation-in-part of application No. 09/113,244, filed on Jul. 10, 1998, now Pat. No. 6,512,162.
(63) Continuation-in-part of application No. 09/316,847, filed on May 21, 1999, now abandoned.
(60) Provisional application No. 60/194,217, filed on Apr. 3, 2000.

(51) Int. Cl. A01H 5/00; C07K 14/52

(52) U.S. Cl. 800/288; 530/351

(57) ABSTRACT

The present invention relates to a process for the production of proteins or polypeptides using genetically manipulated plants or plant cells, as well as to the genetically manipulated plants and plant cells per se (including parts of the genetically manipulated plants), the heterologous protein material (e.g., a protein, polypeptide and the like) which is produced with the aid of these genetically manipulated plants or plant cells, and the recombinant polynucleotides (DNA or RNA) that are used for the genetic manipulation.

### CROSS REFERENCE TO RELATED APPLICATIONS

The present invention is related to and claims the benefit, under 35 U.S.C. §120, of patent applications Ser. Nos. 09/113,244, filed Jul. 10, 1998, U.S. Ser. No. 09/316,847, filed May 20, 1999, and is related to and claims the benefit, under 35 U.S.C. §119(e), of provisional patent application Serial No. 60/194,217, filed Apr. 3, 2000, which are expressly incorporated fully herein by reference.

### FIELD OF INVENTION

This invention describes a novel method of producing and recovering bioactive recombinant proteins from plants. General methods of designing and engineering plants for expression of such proteins, and methods of purification, are also disclosed. Methods for the expression of proteins, such as growth hormone (GH) and granulocyte colony stimulating factor (G-CSF), in plants, and methods of isolating authentic heterologous proteins from plants are specifically disclosed. The new method may be more cost-effective than other large-scale expression systems, by eliminating the need for refolding and other extensive manipulations that generate an active protein with a desired amino terminus.

### BACKGROUND OF THE INVENTION

Recombinant proteins that mimic or have the same structure as native proteins are highly desired for use in therapeutic applications, as components in vaccines and diagnostic test kits, and as reagents for structure/function studies. Mammalian, bacterial, and insect cells are commonly used to express recombinant proteins for such applications. Systems capable of accurately producing the desired protein within the host cell are preferred to systems that generate modified proteins or that require extensive procedures to remove the undesired forms.

Although the biotechnology industry has directed its efforts to eukaryotic hosts like mammalian cell tissue culture, yeast, fungi, insect cells, and transgenic animals, to express recombinant proteins, these hosts may suffer particular disadvantages. For example, although mammalian cells are capable of correctly folding and glycosylating bioactive proteins, the quality and extent of glycosylation can vary with different culture conditions among the same host cells. Yeast, alternatively, produce incorrectly glycosylated proteins that have excessive mannose residues, and generally exhibit limited post-translational processing. Other fungi may be available for high-volume, low-cost production, but they are not capable of expressing many target proteins. Although the baculovirus insect cell system can produce high levels of glycosylated proteins, these proteins are not secreted, however, thus making purification complex and expensive. Transgenic animals are subject to lengthy lead times to develop herds with stable genetics, high operating costs, and contamination by prions or viruses.

Prokaryotic hosts may also suffer disadvantages in expressing heterologous proteins. For example, the post-translational modifications required for bioactivity may not be carried out in the prokaryote host. Some of these post-translational modifications include signal peptide processing, pro-peptide processing, protein folding, disulfide bond formation, glycosylation, gamma carboxylation, and beta-hydroxylation. As a result, complex proteins derived from prokaryote hosts are not always properly folded or processed to provide the desired degree of biological activity. Consequently, prokaryote hosts have generally been utilized for the expression of relatively simple foreign polypeptides that do not require folding or post-translational processing to achieve a biologically active protein. Indeed, the costs associated with the inability of bacteria to perform many of the post-translational modifications required for the biological activity of recombinant proteins of mammals limit the value of this host system. More specifically, extensive post-purification chemical and enzymatic treatments can be required to obtain biologically active protein.

An additional disadvantage associated with expressing recombinant proteins in prokaryotes, such as E. coli, is that the proteins often retain an additional amino acid residue such as methionine at their amino terminus. This methionine residue (encoded by the ATG start codon) is often not present, however, on many native or recombinant proteins harvested from eukaryotic host cells. Thus, the amino termini of many proteins made in the cytoplasm of E. coli must be processed by enzymes, such as methionine aminopeptidase, so that after expression the methionine is cleaved off the N-terminus. Bassat et al., 169 J. Bacteriol. 751-57 (1987).

The amino acid composition of protein termini are biased in many different manners. Berezovsky et al., 12(1) Protein Eng'g 23-30 (1999). Systematic examination of N-exopeptidase activities led to the discovery of the ‘N-terminal’- or ‘N-end rule’: the N-terminal (f)Met is cleaved if the next amino acid is Ala, Cys, Gly, Pro, Ser, Thr, or Val. If this next amino acid is Arg, Asp, Asn, Glu, Gln, Ile, Leu, Lys or Met, the initial (f)Met remains as the first amino acid of the mature protein. The radii of hydration of the amino acid side chains was proposed as physical basis for these observations. Bachmain et al., 234 Science, 179-86 (1986); Varshavsky, 69 Cell, 725-35 (1992). The half-life of a protein (from three minutes to twenty hours), is dramatically influenced by the chemical structure of the N-terminal amino acid. Stewart et al., 270 J. Biol. Chem., 25-28 (1995); Griegoryev et al., 271 J. Biol. Chem., 28521-32 (1996). Site-directed mutagenesis subsequently confirmed the ‘N-end rule’ by monitoring the life-span of recombinant proteins containing altered N-terminal amino acid sequences. Varshavsky, 93 P.N.A.S. 12142-49 (1996). A statistical analysis of the amino acid sequences at the amino termini of proteins suggested that Met and Ala residues are over-represented at the first position, whereas at positions +2 and +5, Thr is preferred. Berezovsky et al., 12(1) Protein Eng'g 23-30 (1999). C-terminal biases, however, show a preference for charged amino acids and Cys residues. Id.

Recombinant proteins that retain the N-terminal methionine, in some cases, have biological characteristics that differ from the native species lacking the N-terminal methionine. Human growth hormone that retains its N-terminal methionine (Met-hHG), for example, may be antigenic compared to hGH purified from natural sources or recombinant hGH that is prepared in such a way that has the same primary sequence as native hGH (lacking an N-terminal methionine). Low-cost methods of generating recombinant proteins that mimic the structure of native proteins are often highly desired for therapeutic applications. Sandman et al., 13 Bio/Tech. 504-06 (1995).

One method of preparing native proteins in bacteria is to express the desired protein as part of a larger fusion protein containing a recognition site for an endoprotease that specifically cleaves upstream from the start of the native amino acid sequences. The recognition and cleavage sites can be those recognized by native signal peptidases, which specifically cleave the signal peptide of the N-terminal end of a protein targeted for delivery to a membrane or for secretion from the cell. In other cases, recognition and cleavage sites can be engineered into the gene encoding a fusion protein so that recombinant protein is susceptible to other non-native endoproteases in vitro or in vivo. The blood clotting factor Xa, collagenase, and the enzyme enterokinase, for example, can be used to release different fusion tags from a variety of proteins. Economic considerations, however, generally preclude use of endoproteases on a large scale for pharmaceutical use. Preparation of hGH from bacterial systems, that encode genes having additional amino acids at the N-terminus are known in the art. U.S. Pat. Nos. 5,633,352; 5,635,604. Derivatives of hGH containing amino acid substitutions are also known. U.S. Pat. No. 5,849,535.

A variety of methods have been described that use one or more exo-peptidases to process the N-terminal amino acids from E. coli-derived recombinant proteins. For example, Met-hGH can be digested by methionine aminopeptidase (MAP) to generate hGH. Additionally, U.S. Pat. Nos. 4,870,017 and 5,013,662 describe the cloning, expression, and use of E. coli methionine aminopeptidase to remove Met from a variety of peptides and Met-IL-2. WO 84/02351 discloses a process for preparing ripe (native) proteins, such as hGH or human proinsulin, from fusion proteins using leucine aminopeptidase. A method of removing the N-terminal methionine from derivatives of human interleukin-2 and hGH using aminopeptidase M, leucine aminopeptidase, aminopeptidase PO, or aminopeptidase P has been described. EP 0 204 527 A1. Aeromonas aminopeptidase (AAP), an exo-peptidase isolated from the marine bacterium A. proteolytica, can also be used to facilitate the release of N-terminal amino acids from peptides and proteins. Wilkes et al., 34(3) Eur. J. Biochem. 459-66, (1973). The sequential removal of N-terminal amino acids from analogs of eukaryotic proteins, formed in a foreign host, by use of Aeromonas aminopeptidase has alos been described. EP 0191827 B1; U.S. Pat. No. 5,763,215.

More complicated methods can also be used to generate recombinant proteins with a native amino terminus. U.S. Pat. No. 5,783,413, for example, describes the simultaneous or sequential use of (a) one or more aminopeptidases, (b) glutamine cyclotransferase, and (c) pyroglutamine aminopeptidase to treat amino-terminally-extended proteins of the formula NH₂-A-glutamine-Protein-COOH to produce a desired native protein.

U.S. Pat. Nos. 5,565,330 and 5,573,923 refers to methods of removing dipeptides from the amino-terminus of precursor polypeptides involving treatment of the precursor with dipetidylaminopeptidase (dDAP) from the slime mold Dictostelium descoideum, which has a mass of about 225 kDa and a pH optimum of about 3.5. Precursors of human insulin, analogues of human insulin, and human growth hormone containing dipeptide extensions were processed by dDAP when the dDAP was in free solution and when it was immobilized on a suitable solid support surface.

The biochemical, technical, and economic limitations on existing prokaryotic and eukaryotic expression systems has created substantial interest in developing new expression systems for the production of heterologous proteins. To that end, plants represent a suitable alternative to other host systems because of the advantageous economics of growing plant crops, plant suspension cells, and tissues such as callus; the ability to synthesize proteins in storage organs like tubers, seeds, fruits and leaves; and the ability of plants to perform many of the post-translational modifications previously described. Strum et al., 175 Planta 170-83 (1988).

Therefore, it is desirable to produce heterologous proteins from a source such as plants, which offer the opportunity for the “Molecular Farming” of important proteins. See, e.g., U.S. Pat. No. 5,550,038. Transgenic plants have been studied over the past several years for potential use in low cost production of high quality, biologically active mammalian proteins. See, e.g., Sijmons et al., 8 Bio/Tech. 217-21 (1990); Vandekerckhove et al., 7 Bio/Tech. 929-32 (1989); Conrad & Fiedler, 26 Plant Mol. Biol. 1023-30 (1994); Ma et al., 268 Sci. 716-19 (1995). Plant-based expression systems may be more cost-effective than other large-scale expression systems for the production of therapeutic proteins, by eliminating the need for refolding, and other extensive manipulations that generate a protein with a native amino terminus. A wide variety of therapeutic proteins, for example, have already been expressed in many different plant hosts. A nonexclusive list of the yield and quality of proteins recovered from transgenic plants is shown in Table 1.

TABLE 1
Expression of heterologous proteins in plants

| Gene | Host | Targeting | Expressed | N-term. | Glycan | Active | Reference |
|---|---|---|---|---|---|---|---|
| interferon | tobacco | secrete | nr | nr | nr | in vitro | U.S. Pat. No. 4,956,282 |
| antibody | tobacco | +/− secrete | 0.8%/ | yes | yes | in vitro | Hein, 7 BIOTECH |
| | leaf | | 0% | | | | PROGRESS 455 |
| | | | | | | | (1991) |
| antibody | tobacco | secrete | nr | nr | yes | mice, | Zeitlin, 16 NAT |
| | cells, soy | | | | | topical | BIOTECH 1361 |
| | | | | | | | (1998) |
| antibody | corn seed | secrete | >3% | yes | yes | in vitro | WO 98/10062 |
| glycan-free | corn seed | secrete | >3% | yes | no | yes | WO 98/10062 |
| antibody | | | | | | | |
| IgA-IgG | tobacco | secrete | 10 μg/ml | nr | likely | in vitro | Ma, 24 EUR J |
| hybrid | leaf | | | | | | IMMUNOL 131 |
| | | | | | | | (1994) |
| scFV | tobacco | +/− secrete | 0.01/0% | nr | nr | in vitro | Schouten, 20 |
| | leaf | | | | | | PLANT MOL BIO |
| | | | | | | | 781 (1996) |
| scFV | tobacco | +/− KDEL | 1/0.01% | nr | nr | in vitro | Schouten, 1996 |
| | leaf | | | | | | |
| insulin | tobacco | secrete | positive | nr | nr | nr | EP 0437320 |
| | leaf | | | | | | |
| insulin | potato | secrete +/− | 0.1/0.05% | nr | nr | no | Arakawa, 16 NAT |
| | tuber | cholera fusion | | | | | BIOTECH 934 |
| | | | | | | | (1998) |
| erythro- | tobacco | secrete | 0.003% | nr | yes | no | Matsumoto 27 |
| poetin | cells | | | | | | PLANT MOL BIO |
| | | | | | | | 1163 (1995) |
| GM-CSF | tobacco | secrete | 0.26 ug/ml | nr | nr | cells | GANZ, |
| | seed | | | | | | TRANSGENIC |
| | | | | | | | PLANTS 281 |
| | | | | | | | (1996) |
| trout | tobacco | secrete | 0.1% | nr | yes | nr | Bosch, 3 |
| growth | | | | | | | TRANSGENIC RES. |
| factor | | | | | | | 304 (1994) |
| human | potato, | secrete | 0.02% | yes | nr | nr | Sijmons 8 |
| serum | tobacco | | | | | | BIO/TECH 217 |
| albumin | | | | | | | (1990) |
| avidin | corn seed | secrete | 3% | yes | yes | in vitro | Hood, 3 PLANT |
| | | | | | | | MOL BIO 291 |
| | | | | | | | (1997) |
| GUS | tobacco | cytosol +/− | 10x activity | nr | nr | yes | Garbarino, 24 |
| | leaf | ubiquitin | | | | | PLANT MOL BIO |
| | | | | | | | 119 (1994) |
| hirudin | canola | secrete + | 1% tsp | nr | nr | in vitro | Parmenter, 29 |
| | seed | oleosin | | | | | PLANT MOL BIO |
| | | | | | | | 1167 (1995); U.S. |
| | | | | | | | Pat. No. 5,650,554 |
| BT toxin | tobacco | +/− plastid | 1%/0.1% | nr | nr | nr | Wong, 20 PLANT |
| | | targeting | | | | | MOL BIO 81 |
| | | | | | | | (1992) |
| hGH | tobacco | secrete | 0.16% | yes | nr | nr | Leite, 1999 |
| | seed | | | | | | |

nr = not reported

The present invention contemplates producing bioactive cytokines from a plant host systems. The cytokines of the present invention may be any mammalian soluble protein or peptide which acts as a humoral regulator at the nano- to pico-molar concentration, and which either under normal or pathological conditions, modulate the functional activities of individual cells and tissues. Furthermore, the cytokines may also mediate interactions between cells directly and regulate processes taking place in the extracellular environment. The cytokines of the present invention belong to the cytokine superfamalies, which include, but are not limited to: the Tumor Growth Factor-beta (TGF-beta) superfamily (comprising various TGF-beta isoforms, Activin A, Inhibins, Bone Morphogenetic Proteins (BMP), Decapentaplegic Protein (DPP), granulocyte colony stimulating factor (G-CSF), Growth Hormone (GH) (including human growth hormone (hGH)), Interferons (IFN), and Interleukins (IL)); the Platelet Derived Growth Factor (PDGF) superfamily (comprising VEGF); the Epidermal Growth Factor (EGF) superfamily (comprising EGF, TGF-alpha, Amphiregulin (AR), Betacellulin, and HB-EGF); the Vascular Epithelial Growth Factor (VEGF) family; Chemokines; and Fibroblast Growth factors (FGF). The methods of the present invention are applicable to any cytokine, whether or not yet discovered, and are not limited to any particular cytokine exemplified herein. See, e.g., Hill et al., 90 P.N.A.S. 5167-71 (1993).

More efficient strategies to process amino acids from the amino terminus of recombinant proteins, such as cytokines including GH, hGH and G-CSF, are desirable to reduce the cost of generating therapeutic proteins that mimic the structure of native proteins. Methods that increase the levels of expression or facilitate the downstream processing of recombinant proteins will also accelerate the selection and development of small chemical molecules and other protein-based molecules destined for large scale clinical trials. Therefore, the method and compositions provided by the present invention may yield more efficient and cost effective means for producing therapeutic proteins that mimic the structure of authentic proteins.

Other objectives, features and advantages of the present invention will become apparent from the following detailed description. The detailed description and the specific examples, while indicating specific embodiments of the invention, are provided by way of illustration only. Accordingly, the present invention also includes those various changes and modifications within the spirit and scope of the invention that may become apparent to those skilled in the art from this detailed description.

### SUMMARY OF THE INVENTION

The present invention provides methods for producing a cytokine in a plant host system in which the plant host system had been transformed with a chimeric nucleic acid that encodes the cytokine, the method including cultivating the transformed plant under conditions that result in the expression of the cytokine in the plant host system. A further aspect of this method includes the purification of the cytokine from the plant host system. According to the method of this invention, the cytokine produced in the plant host system is free from amino acid modifications such as hydoxyproline, and free from novel glycosylations.

The method of the present invention employs a chimeric nucleic acid sequence that includes a first nucleic acid that regulates the transcription in the plant host system of a second nucleic acid sequence that encodes a signal sequence that is linked in reading frame to a third nucleic acid sequence that encodes a cytokine. In a preferred aspect of the invention, the chimeric nucleic acid sequence also contains a fourth nucleic acid sequence. In a more preferred aspect of the invention, the fourth nucleic acid is a KDEL amino acid sequence. In another preferred aspect of the invention, the first nucleic acid is a plant-active transcription promoter. In another preferred aspect of the invention, the second nucleic acid sequence targets the cytokine to a sub-cellular location within the plant host system. Such sub-cellular locations are preferably the cytosol, plastid, or endoplasmic reticulum. In another preferred aspect of the method of this invention, the second nucleic acid encodes a portion of ubiquitin, more preferably a monomer of yeast ubiquitin gene or a monomer of potato ubiquitin gene 3. In another preferred aspect of the method, the second nucleic acid encodes a portion of the oleosin sufficient to provide sub-cellular targeting. In a still more preferred aspect of the invention, the oleosin portion is specifically cleavable by enzymatic or chemical means included between the oleosin portion and the cytokine. In a preferred aspect of the invention, the nucleic acid sequence encoding oleosin is derived from soy.

The method of the present invention provides for the production in a plant host system of cytokines such as those of the cytokine superfamilies TGF-beta, PDGF, EGF, VEGF, chemokines, and FGF. More preferably, the cytokine is either GH, hGH, or G-CSF.

The invention described herein also provides a plant host system that has been transformed with a chimeric nucleic acid sequence that includes a first nucleic acid that regulates the transcription in the plant host system of a second nucleic acid sequence that encodes a signal sequence that is linked in reading frame to a third nucleic acid sequence that encodes a cytokine. In a preferred embodiment of the plant host system, the chimeric nucleic acid sequence also contains a fourth nucleic acid sequence. In a more preferred embodiment of the invention, the fourth nucleic acid is a KDEL amino acid sequence. In another preferred embodiment of the invention, the first nucleic acid is a plant-active transcription promoter. In another preferred aspect of the plant host system, the second nucleic acid sequence targets the cytokine to a sub-cellular location within the plant host system. Such sub-cellular locations are preferably the cytosol, plastid, or endoplasmic reticulum. In another preferred embodiment of this invention, the second nucleic acid encodes a portion of ubiquitin, more preferably a monomer of yeast ubiquitin or a monomer of potato ubiquitin gene 3. In another preferred embodiment, the second nucleic acid encodes a portion of the oleosin gene sufficient to provide sub-cellular targeting. In a still more preferred embodiment of the invention, the oleosin portion is specifically cleavable by enzymatic or chemical means included between the oleosin portion and the cytokine. In yet another a preferred embodiment, the nucleic acid sequence encoding oleosin is derived from soy.

Additionally, the plant host system of the present invention provides for the production in a plant host system of cytokines such as those of the cytokine superfamilies TGF-beta, PDGF, EGF, VEGF, chemokines, and FGF. More preferably, the cytokine is either GH, hGH, or G-CSF. Moreover, the cytokine may be purified from the plant host system, and the cytokine produced in the plant host system is free from amino acid modifications such as hydoxyproline, and free from novel glycosylations.

The present invention also relates to a chimeric nucleic acid sequence expressed in a plant host system, that includes a first nucleic acid that regulates the transcription in the plant host system of a second nucleic acid sequence that encodes a signal sequence that is linked in reading frame to a third nucleic acid sequence that encodes a cytokine. In a preferred embodiment of the invention, the chimeric nucleic acid sequence also contains a fourth nucleic acid sequence. In a more preferred embodiment of the invention, the fourth nucleic acid is a KDEL amino acid sequence. In another aspect of the invention, the first nucleic acid is a plant-active transcription promoter. In another preferred aspect of the chimeric nucleic acid sequence, the second nucleic acid sequence targets the cytokine to a sub-cellular location within the plant host system. Such sub-cellular locations are preferably the cytosol, plastid, or endoplasmic reticulum. In another preferred aspect of the invention, the second nucleic acid encodes a portion of ubiquitin, more preferably a monomer of yeast ubiquitin or a monomer of potato ubiquitin gene 3. In another preferred embodiment, the second nucleic acid encodes a portion of the oleosin gene sufficient to provide sub-cellular targeting. In a still more preferred embodiment of the chimeric nucleic acid, the oleosin portion is specifically cleavable by enzymatic or chemical means included between the oleosin portion and the cytokine. In yet another a preferred embodiment, the nucleic acid sequence that encodes oleosin is derived from soy.

In a preferred embodiment of the invention, the chimeric nucleic acid sequence provides for the production in a plant host system of cytokines such as those of the cytokine superfamilies TGF-beta, PDGF, EGF, VEGF, chemokines, and FGF. More preferably, the cytokine is either GH, hGH, or G-CSF. In another preferred embodiment of the invention, the hGH encoded by a portion of the chimeric nucleic acid sequence has an authentic N-terminus. In another preferred embodiment, the G-CSF encoded by a portion of the chimeric nucleic acid sequence has a authentic N-terminus. Preferrably, the cytokines encoded by the chimeric nucleic acid sequences are free of novel glycosylations and modified amino acids such as hydroxyproline. In another preferred embodiment of the invention, the chimeric nucleic acid sequence is included in an expression cassette.

The invention embodied herein also contemplates a plant, plant cell culture, or plant seed transformed with this chimeric nucleic acid sequence. The invention herein also contemplates a cytokine produced in a plant that has been transformed by the chimeric nucleic acid sequence described herein.

The invention herein provides a method for preparing a bioactive, authentic mammalian growth hormone in corn plants, by inserting a gene for said growth hormone into a corn plant expression vector; transforming corn plant cells with an expression vector; generating whole corn plants from the transformed corn cells; harvesting corn seed from whole corn plants; and purifying the growth hormone from powdered corn seed. In another aspect of the invention, corn plants and corn seed have been prepared by this method. In a most preferred aspect of this method, the mammalian growth hormone is human growth hormone. In another aspect of this method, the growth hormone accumulates to a level greater than 1% of the total soluble protein in a plant sample. More particularly, the growth hormone accumulates to level greater than 5% of the total soluble protein in a plant sample. In another preferred aspect of the method, the growth hormone is not glycosylated. In yet another preferred embodiment of the method, the corn plant expression vector is pwrg4825.

In yet another aspect of the method of the invention, authentic human growth hormone from corn seed is further purified by extracting corn seed (that has been crushed or powdered) with buffered saline, wherein said extraction is carried out at a pH ranging from about pH 8 to about pH 10; adding urea to a concentration of about 2M to 3.5 M urea; adjusting the pH of the extract to about pH 5; clarifying the solution; purifying by cation exchange chromatography, wherein said cation exchange chromatography is carried out in the presence of urea at a pH from about 4.5 to about 5.5; and purifying by anion exchange chromatography, wherein said anion exchange chromatography is carried out in the absence of urea at a pH from about 7.0 to about 8.0.

### BRIEF DESCRIPTION OF THE DRAWINGS

FIG. 1 depicts the amino acid sequence of hGH, a single-chain polypeptide (22 kDa) (SEQ ID NO:12), containing four cysteine residues involved in two disulfide bond linkages.

FIG. 2 is a diagram of the corn transformation vector pwrg4825. Restriction sites used for the construction are shown. Plant expression elements are defined as boxes, and bacterial vector sequences as a thin line.

FIG. 3 is a chart summarizing different vectors constructed for the expression of hGH in plants.

FIG. 4 is a Western blot of hGH transient expression (using CaMV 35S, or eFMV for CTP2) with different targeting signals: extensin, targeting secretion (EXT); ′5 UTR, targeting cytosol (DSSU); chloroplast transit peptide, targeting plastids (CTP2); and hGH control (Stnd).

FIG. 5 shows a Western blot of hGH fexpressed transiently in soy hypocotyl tissues from vectors with the CaMV 35S promoter and different targeting signals: standard (3 ng); null (—); cytosol (DSSU); extensin (EXT); potato ubiquitin (potato ubi); and yeast ubiquitin (yeast ubi).

FIG. 6 shows a Western blot of an hGH oleosin fusion expressed transiently in soy hypocotyl tissues: null (—); standard (1 ng); oleosin fusion (OLE); and extensin (EXT).

FIG. 7 is a chart summarizing the expression of hGH in transgenic soy seeds.

FIG. 8 depicts a Western blot of hGH expression in transgenic soy seeds (A, B, C, and D, two seeds each from 2 different pods) compared to standards (1 ng and 0.2 ng).

FIG. 9 charts a summary for transgenic tobacco cell and suspension media expression of hGH with different targeting designs.

FIG. 10 is a Western blot showing hGH expression with different targeting signal sequences in tobacco cells: cytosol; endoplasmic reticulum (ER); plastid; null (N); and standard (32 ng).

FIG. 11 summarizes tobacco plant expression of hGH with different targeting designs.

FIG. 12 depicts the bioactivity of hGH secreted and partially purified from transformed tobacco cells compared to an E. coli standard.

FIG. 13 plots the mass spectrometry results for Phe-hGH expressed in tobacco cells.

FIG. 14 tabulates the corn seed expression and inheritance of different hGH transformation events.

FIG. 15 is a Western blot comparing hGH expression found in seed extracts from independent first-generation transformation events, compared to a 0.5 ng hGH standard spiked into a non-expressing seed extract.

FIG. 16 depicts graphically the bioactivity of corn seed-derived hGH (Corn sample) compared with that of refolded E. coli-derived hGH in null corn extract (spiked control). Samples were diluted, and tested via a cell proliferation-based assay, to show bioactivity at a level expected from the ELISA-based quantitation.

FIGS. 17A-B presents mass spectrophotometry data of corn-derived hGH. Corn seed hGH was purified, and analyzed by mass spectrophotometry to show recovery of significant levels of authentic-sized hGH at 21,225 Da, consistent with proper disulfide linkages and no deleterious amino acid modifications.

FIG. 18 shows a scheme for isolating human growth hormone from corn seed.

FIGS. 19A-B illustrates anion exchange HPLC of hGH isolated from corn seed and E. coli. FIG. 19A shows an anion exchange HPLC profile of hGH isolated from corn seed. FIG. 19B shows the profile of hGH isolated from E. coli.

FIG. 20 shows the reverse-phase HPLC profile of hGH isolated from corn seed and E. coli. Panel A shows a reverse-phase HPLC profile of hGH isolated from corn seed. Panel B shows the profile of hGH isolated from E. coli.

FIGS. 21A-B depicts the tryptic peptide reverse phase HPLC chromatograms of hGH isolated from corn seed (A) and E. coli(B).

FIG. 22 compares graphically the weight gain in rats treated with either corn-derived or E. coli-derived hGH.

FIG. 23 charts the vectors designed for the expression of G-CSF.

FIG. 24 is a Western blot showing the transient expression (via the CaMV 35S promoter or eFMV promoter for CTP)of MetAla-GCSF targeted to different subcellular organelles of soy and corn tissues.

FIG. 25 is a Western blot reflecting transient expression of G-CSF in corn leaves, comparing different codon designs and non-transformed leaves against a 10 ng standard.

FIG. 26 is a Western depicting transient expression of G-CSF in corn, with (+KDEL) and without the KDEL (−KDEL) fusion, comparing total corn extract (total) to extracellular wash (wash), and a 5 ng standard.

FIG. 27 presents a summary of G-CSF expression in tobacco cells and suspension media.

FIG. 28 shows a Western blot of G-CSF expressed in transgenic tobacco cells and resultant suspension media, from different constructs. All constructs contained a secretion signal, but differ in codon design and use of KCEL fusion.

FIG. 29 illustrates the results of electron spray mass spectrometry of purified MetAla G-CSF.

FIG. 30 charts the results for liquid chromatography-electron spray mass spectrometry analysis of partially digested purified MetAla G-CSF.

FIG. 31 illustrates the results of a bioassay of plant-derived (tobacco cell) MetAla G-CSF compared to an E coli derived refolded standard.

### DETAILED DESCRIPTION OF THE INVENTION

It is understood that the present invention is not limited to the particular methodology, protocols, cell lines, vectors, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a cytokine” is a reference to one or more cytokines and includes equivalents thereof known to those skilled in the art and so forth. Indeed, one skilled in the art can use the methods described herein to produce any cytokine (known presently or subsequently) in plant host systems.

Transgenic plants have been studied for several years for potential use in low-cost production of high quality, biologically active mammalian proteins. For example human serum albumin (HSA), has been successfully secreted into the medium from plant cells derived from both potato and tobacco plants. Sijmons et al., 8 Bio/Tech. 217-21 (1990). Additionally, various other proteins have been successfully produced in plants. See, e.g., Kusnadi et al., 56(5) Biotech. & Bioeng'g 473-84 (1997); U.S. Pat. No. 5,550,038. Human serum albumin, transgenic plant rabbit liver cytochrome P450, hamster 3-hydroxy-3-methylglutaryl CoA reductase, and the hepatitis B surface antigen have been reported in the art. See, e.g., Sijmons,1990; Saito et al., 88 P.N.A.S. 7041-45 (1991); Mason et al., 89 P.N.A.S. 11745-49 (1992). Additionally, low level expression of murine GM-CSF has been reported in tobacco cell suspension culture, although the protein was not characterized. Li et al., 7(6) Mol. Cells 783-787 (1997).

Additionally, expression of monoclonal antibodies in plant host systems has been widely studied primarily due to their potential value as therapeutic and clinical reagents. See Düring, Inaugural Dissertation (1988); Düring & Hippe, 370 Biol. Chem. Hoppe Seyler 888 (1989); Düring et al., 15 Plant Mol. Biol. 281-93 (1990). These plant host systems include Nicotania tabacum (tobacco) plants, capable of expressing IgG antibodies. Hiatt et al., 342 Nature 76-78 (1989); Ma et al., 24 Eur. J. Immunol. 131-38 (1994); U.S. Pat. Nos. 5,202,422 and 5,639,947. More recently, a more complex IgA antibody was synthesized in transgenic tobacco plants. U.S. Pat. No. 5,959,177. The synthesis of IgA in rice has been reported recently as well. WO 99/66,026. Antibodies expressed in Zea mays (corn) plants include monoclonal antibody BR96 and monoclonal antibody NeoR×451 (WO 98/10,062).

Single-chain antibody fragments are well-known in the art. Bird et al., 242 Sci. 423-26 (1988). Functional single chain fragments have been successfully expressed in the leaves of tobacco and Arabidopsis plants. Owen et al. 10 Bio/Tech. 790-94 (1992); Artsaenko et al., 8 Plant J. 745-50 (1995); Fecker et al., 32 Plant Mol. Biol. 979-86 (1996). Long term storage of single chain antibody fragments has also been indicated in tobacco seeds. Fielder et al. 13 Bio/Tech. 1090-93 (1995). L6 sFv single chain anti-carcinoma antibody, anti-TAC sFv (that recognizes L2 receptor) and G28.5 sFv single-chain antibody (that recognizes CD40 cell surface protein) have been produced in high levels in tobacco culture. U.S. Pat. No. 6,080,560. Additionally, the single-chain antibody L6 has been successfully produced in corn and soy. Cooley et al., 108(2) Plant Physiol. 50 (1995).

As discussed above, most transgenic plant expression studies have been performed in tobacco leaves. Observations in tobacco leaves, however, may not extend to other host species or tissue types. In most cases, the level of the desired protein is usually below 1% of the total soluble protein. The quality of the expressed protein is often not confirmed by N-terminal sequence analysis and the glycosylation state of each protein often remain unexamined. Novel glycosylation events, such as O-linked glycosylation, if they occur, may be overlooked.

In the broadest aspect, the present invention provides methods and compositions for producing and recovering bioactive recombinant proteins from plants. In a preferred aspect of the present invention, recombinant proteins include cytokines. The cytokines of the present invention may be any mammalian soluble protein or peptide which acts as a humoral regulator at the nano- to pico-molar concentration, and which either under normal or pathological conditions, modulate the functional activities of individual cells and tissues. Furthermore, the cytokines may also mediate interactions between cells directly and regulate processes taking place in the extracellular environment. The cytokines of the present invention are belong to the cytokine superfamalies, which include, but are not limited to: the Tumor Growth Factor-beta (TGF-beta) superfamily (comprising various TGF-beta isoforms, Activin A, Inhibins, Bone Morphogenetic Proteins (BMP), Decapentaplegic Protein (DPP), G-CSF, Growth Hormone (GH, more particularly human growth hormoner (hGH)), Interferons (IFN), and Interleukins (IL)); the Platelet Derived Growth Factor (PDGF) superfamily (comprising VEGF); the Epidermal Growth Factor (EGF) superfamily (comprising EGF, TGF-alpha, Amphiregulin (AR), Betacellulin, and HB-EGF); the Vascular Epithelial Growth Factor (VEGF) family; Chemokines; and Fibroblast Growth factors (FGF). See, e.g., Hill et al., 90 P.N.A.S. 5167-71 (1993).

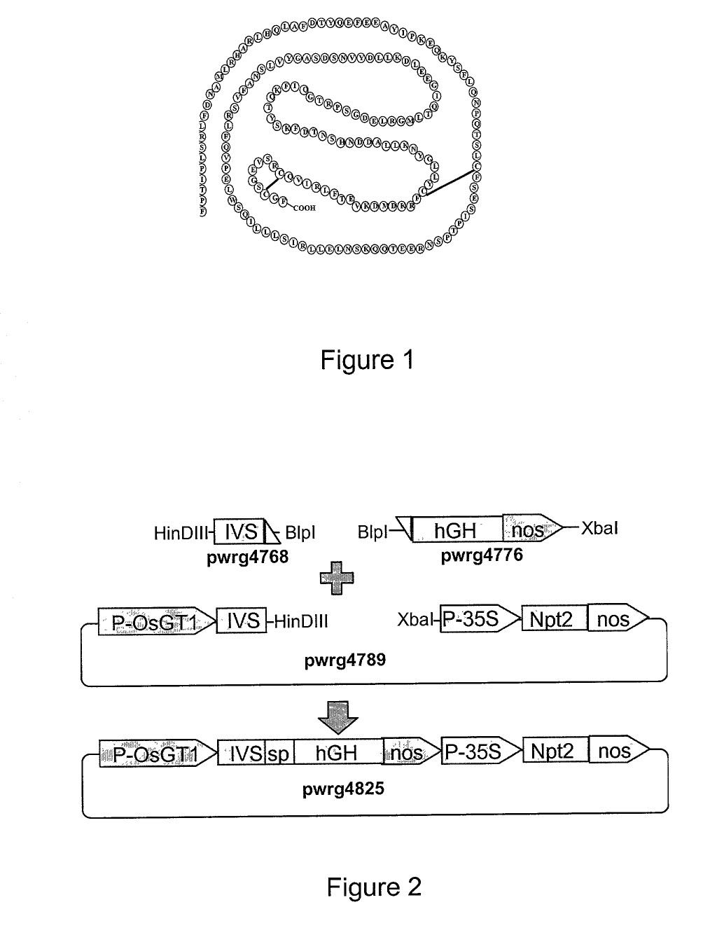

A preferred aspect of the present invention relates to the production of bioactive, authentic growth hormone (GH) from a plant host system. A preferred GH is human growth hormone (hGH). This hormone, depicted in FIG. 1, is a single chain polypeptide hormone of 191 amino acids (SEQ ID NO:12) produced mainly by the adenohypophysis (anterior pituitary), but is also expressed in mature lymphocytes. Growth hormone (also called somatotropin) is released in response to the hypothalamus-derived GH releasing hormone. The physiological effect of hGH is the promotion of bone growth, cartilage, and soft tissues. Overproduction of hGH leads to acromegaly, while a deficiency in hGH may result in dwarfism. In addition, hGH also functions in the maintenance of lean body mass, and the regulation of the synthesis of other hormones, such as Insulin-like Growth Factor-1 (IGF-1). Growth Hormone, Cytokines Online Pathfinder Encyclopedia (<http://www.copewithcytokines.de/>).

There have been several attempts to express growth hormone derivatives in plants. A genomic hGH gene was inserted into plant cells, but the gene was not effectively processed and expression was not examined. Barta, 6 Plant Mol. Biol 347-57 (1986). The distantly-related trout growth hormone (tGH-II) fused to a plant signal peptide, however, was expressed in plants. Bosch et al., 3 Transgen. Res. 304-10 (1994). Partial glycosylation was observed in tobacco leaves, with levels below ≦0.1% of the total soluble protein, for constructs containing a plant signal peptide. Bosch, 1994. No expression was observed in Arabidopsis seed using a seed-specific promoter. Liete, Int'l. Mol. Farming Conference, London, Ontario (Aug. 29, 1999). Liete reported that the hGH gene, when fused to a plant signal peptide, hGH accounted for less than ≦0.16% of the total soluble protein in tobacco seed. Id. The protein had the expected amino acid sequence and was active in receptor binding assays.

Futhermore, non-nuclear, tobacco plastid transformation for expression of hGH has been described. Staub et al., 18 Nature BioTech. 333-38 (2000). Staub reported that both non-natural methionine and ubiquitin fusions yielded expression in leaves ranging from 0.2-7% of the total soluble protein. The ubiquitin fusion showed activity, and some material of the correct mass, indicating no glycosylation and correct N-terminus. Nuclear transformation showed expression lower than 0.03% for either secreted or chloroplast-targeted proteins, with no other data presented.

Additionally, recovery of active somatotropin prepared from corn plants has been reported, but the type of somatotropin, transformation details, expression levels, and protein quality were not discussed. White, Conference on Transgenic Prod. Of Human Therapeutics, Waltham, Mass (1998).

The present invention also contemplates producing biologically active, authentic granulocyte colony stimulating factor (G-CSF) from a plant host system. G-CSF is an O-glycosylated 19 kDa glycoprotein, and the biologically active form is a monomer. cDNA analysis of G-CSF has revealed a protein of 207 amino acids containing a hydrophobic secretory signal sequence of 30 amino acids. Furthermore, G-CSF contains 5 cysteine residues, four of which form disulfide bonds. The sugar moiety of G-CSF is not required for full biological activity. G-CSF, Cytokines Online Pathfinder Encyclopedia (<http://www.copewithcytokines.de/>). A particular therapeutic product is produced from mammalian cells, with 174 amino acids, the native N-terminus and mammalian-type O-glycosylation. Ono et al., 30A(3) Eur. J. Cancer S7-S11 (1994). A product is also produced from bacterial cells, with 175 amino acids, a non-native methionine at the N-terminus, and no glycosylation. Physician's Desk Reference (2000).

G-CSF, is used in the treatment of transient phases of leukopenia that may follow chemotherapy and/or radiotherapy. It is also used to enhance immune system deficiency caused by diseases such as AIDS. G-CSF has been shown to expand the myleoid cell lineage. Thus, pretreatment with recombinant human G-CSF prior to bone marrow harvest can improve the graft by increasing the total number of myeloid lineage restricted progenitor cells. This may result in a stable, but not accelerated, myeloid engraftment of autologous marrow. Id.

In accordance with the present invention, methods and materials are provided for modifying expression vector design to increase yield and improve quality of cytokines expressed in a plant host system. The present invention contemplates optimizing expression vector design by modifying promoters, 5′UTRs, signal sequences, structural genes, and 3′UTRs. The design parameters of the present invention may include, but are not limited to codon usage, primary transcript structure, translational enhancing sequences, appropriate use of intron splice sites, RNA stabilizing, RNA destabilizing/processing sequences.

In a further aspect, N- or C-terminal fusions may also be established to facilitate optimal yield, quality, and protein processing. The present invention contemplates the recombinant cytokine fused to signal peptides, such as ubiquitin, soy oleosin oil binding protein, and extensin, to (1) target the expressed cytokine to specific sub-cellular locations within the plant host system, (2) enhance product accumulation and quality, and (3) provide a means for simple recovery of the recombinant cytokine from the plant host system.

Furthermore, the present invention envisions the C-terminus of the recombinant cytokine fused to a stabilizing element, such as the KDEL sequence, to enhance recombinant cytokine accumulation. In an additional aspect, a protease site or self-processing site may be included to facilitate the release of the signal peptide or stabilizing element from the recombinant cytokine.

In accordance with further embodiments of the present invention, methods and materials are provided for a novel means of the production of cytokines that can be easily purified from a plant host system by optimizing expression vector design. The expression vector design may be modified to maximize RNA transcription and translation (protein expression), protein targeting (e.g., nucleus, plastid, cytosol, endoplasmic reticulum), protein modification and fusion, protein expression in different plant tissues, and protein expression in different plant species.

In accordance with one aspect of the present invention, methods and materials are provided for a novel means of production of recombinant cytokines in a plant host system that are easily separated from other host cell compartments. Purification of the recombinant cytokine is greatly simplified by this approach. The recombinant nucleic acid encoding the cytokine may be part of all of a naturally occurring DNA sequence from any source, it may be a synthetic DNA sequence or it may be a combination of naturally occurring and synthetic sequences. The present invention includes the steps, singly or in sequence, of preparing an expression vector that includes a first nucleic acid sequence that regulates the transcription of a second nucleic acid sequence encoding a significant portion of a peptide that targets a protein to a sub-cellular location, and, fused to this second nucleic acid, a third nucleic acid encoding the cytokine of interest; generating a transformed plant host system in which the cytokine of interest is expressed; and purifying the cytokine of interest from the transgenic plant host system.

In one aspect of the present invention, the first nucleic acid sequence may comprise a plant active promoter, such as the CaMV 35S promoter, the second nucleic acid sequence may comprise additional 5′ regulatory sequences, and the third nucleic acid sequence may comprise the cytokine of interest . The 5′ regulatory sequences may contain signal sequences which target the cytokine to a specific sub-cellular location within the plant host system. In one preferred embodiment of the present invention, a nucleic acid sequence encoding a cytokine of interest may be fused with a 5′ regulatory sequence allowing significant accumulation of the mature cytokine in the cytosol. In another embodiment of the present invention, the nucleic acid sequence encoding the cytokine of interest may be fused to a 5′ regulatory sequence containing a signal peptide that targets the cytokine of interest to the endoplasmic reticulum. In yet another preferred embodiment of the present invention, the nucleic acid sequence encoding the cytokine of interest may be fused with a 5′ regulatory sequence that targets the cytokine of interest to the plastid. Targeting the mature cytokine to a specific sub-cellular location may result in increased accumulation of the cytokine and easier purification of the cytokine from the plant host system.

In accordance with another aspect of the present invention, a plant host system is contemplated that has already been transformed with an expression vector comprising a first nucleic acid sequence that regulates the transcription of a second nucleic acid sequence encoding a significant portion of a peptide that targets a protein to a sub-cellular location and fused to this second nucleic acid, a third nucleic acid encoding the cytokine of interest. Another aspect of this embodiment of the present invention comprises cultivating the plant host system under the appropriate conditions to facilitate the expression of the recombinant cytokine, and purifying the recombinant cytokine from the plant host system.

In accordance with yet another aspect of the present invention, methods and materials are provided to improve the quality of the recombinant cytokine produced in a plant host system. The present invention contemplates generating a recombinant cytokine that has a methionine-free N-terminus that is identical to the natural N-terminus of the mature cytokine. Furthermore, the present invention envisions producing a recombinant cytokine in a plant host system that is free from novel glycosylations and amino acid modifications (such as hydroxyproline).

In a specific embodiment of the present invention, a fusion protein is generated consisting of the N-terminus of the recombinant cytokine and ubiquitin. The ubiquitin-cytokine fusion causes the expression of the fusion protein containing the ubiquitin gene at the 5′ end, and subsequent in vivo processing cleaves the ubiquitin region from the recombinant cytokine, resulting in a cytokine free of both ubiquitin and methionine at the N-terminus.

In an additional embodiment of the present invention, a fusion protein is generated comprising a region of the soy oleosin oil binding protein, a protease site, and the cytokine of interest. This fusion protein ultimately results in a mature cytokine that is free of the oleosin/protease fusion and a methionine N-terminus.

The transformed plant host system of the present invention may be any monocotyledonous or dicotyledonous plant or plant cell. The monocotyledonous plants include, but are not limited to, corn, cereals, grains, grasses, and rice. The dicotyledonous plants may include, but are not limited to, tobacco, tomatoes, potatoes, and legumes including soybean and alfalfa.

### Definitions

Amino acid sequences: as used herein, includes an oligopeptide, peptide, polypeptide, or protein sequence, and fragment thereof, and to naturally occurring or synthetic molecules.

Asexual propagation: producing progeny by regenerating an entire plant from leaf cuttings, stem cuttings, root cuttings, single plant cells (protoplasts) and callus.

Authentic: as used herein, means of the desired or natural form, being properly folded, having the proper disulfide bonds or other post-translational improvements, with no undesired post-translational modifications.

Bioactive: as used herein, means displaying a measurable response by a cell, tissue, organ or organism.

Chemical derivative: as used herein, a molecule is said to be a “chemical derivative” of another molecule when it contains additional chemical moieties not normally a part of the molecule. Such moieties can improve the molecule's solubility, absorption, biological half-life, and the like. The moieties can alternatively decrease the toxicity of the molecule, eliminate or attenuate any undesirable side effect of the molecule, and the like.

Dicotyledon (dicot): a flowering plant whose embryos have two seed halves or cotyledons. Examples of dicots include: tobacco; tomatoes; potatoes, the legumes including alfalfa and soybeans; oaks; maples; roses; mints; squashes; daisies; walnuts; cacti; violets; and buttercups.

Enhancers

Enhancer sites, which are standard and known to those in the art, may be included in the expression vectors to increase and/or maximize transcription of the cytokine of interest in a plant host system. These include, but are not limited to, peptide export signal sequences, optimized codon usage, introns, polyadenylation, and transcription termination sites. Methods of modifying nucleic acid constructs to increase expression levels in plants are also generally known in the art. See, e.g Rogers et al., 260 J. Biol. Chem. 3731-38 (1985); Cornejo et al., 23 Plant Mol. Biol. 567-81 (1993).

In engineering a plant system that affects the rate of transcription of a cytokine, various factors known in the art including regulatory sequences such as positively or negatively acting sequences, enhancers and silencers, as well as, chromatin structure can affect the rate of transcription in plants. The present invention provides that at least one of these factors may be utilized in engineering plants to express a cytokine of interest.

Fragments: include any portion of an amino acid sequence which retains at least one structural or functional characteristic of the subject post-translational enzyme or heterologous polypeptide.

Functional equivalent: a protein or nucleic acid molecule that possesses functional or structural characteristics that are substantially similar to a heterologous protein, polypeptide, enzyme, or nucleic acid. A functional equivalent of a protein may contain modifications depending on the necessity of such modifications for the performance of a specific function. The term “functional equivalent” is intended to include the “fragments,” “mutants,” “hybrids,” “variants,” “analogs,” or “chemical derivatives” of a molecule.

Fusion protein: a protein in which peptide sequences from different proteins are covalently linked together.

Introduction: insertion of a nucleic acid sequence into a cell, by methods including infection, transfection, transformation or transduction.

Isolated: as used herein, refers to any element or compound separated not only from other elements or compounds that are present in the natural source of the element or compound, but also from other elements or compounds and, as used herein, preferably refers to an element or compound found in the presence of (if anything) only a solvent, buffer, ion, or other component normally present in a solution of the same.

Monocotyledon (monocot): a flowering plant whose embryos have one cotyledon or seed leaf. Examples of monocots include: lilies; grasses; corn; rice, grains including oats, wheat and barley; orchids; irises; onions and palms.

Operably linked: as used herein, refers to the state of any compound, including but not limited to deoxyribonucleic acid, when such compound is functionally linked to any promoter.

Plant culture medium: any combination of amino acids, salts, sugars, plant growth regulators, vitamins, and/or elements and compounds that will maintain and/or support the growth of any plant, plant cell, or plant tissue. A typical plant culture medium has been described by Murashige & Skoog, 15 Physiol. Plant. 473-97 (1962).

Plant host system: includes plants, including, but not limited to, monocots, dicots, and specifically maize, soybean, and tobacco. Plant host system also encompasses plant cells. Plant cells includes suspension cultures, embryos, merstematic regions, callus tissue, leaves, roots, shoots, gametophytes, sporophytes, pollen, seeds and microspores. Plant host systems may be at various stages of maturity and may be grown in liquid or solid culture, or in soil or suitable medium in pots, greenhouses or fields. Expression in plant host systems may be transient or permanent. Plant host system also refers to any clone of such a plant, seed, selfed or hybrid progeny, propagule whether generated sexually or asexually, and descendents of any of these, such as cuttings or seed.

Plant sample: a tissue, organ, or subset of the plant, selected to have the preferred accumulation level, quality, or storability for production of the desired protein.

Plant transformation and cell culture: broadly refers to the process by which plant cells are genetically altered and transferred to an appropriate plant culture medium for maintenance, further growth, and/or further development.

Promoters

To produce the desired protein expression in plants, the expression of the heterologous protein may be under the direction of a plant promoter. Promoters suitable for use in accordance with the present invention are described in the art. See e.g., WO 91/198696. Examples of promoters that may be used in accordance with the present invention include non-constitutive promoters or constitutive promoters, such as, the nopaline synthetase and octopine synthetase promoters, cauliflower mosaic virus (CaMV) 19S and 35S promoters, and the figwort mosaic virus (FMV) 35 promoter. See U.S. Pat. No. 6,051,753.

In one aspect of the present invention, the cytokine of interest may be expressed in a specific tissue, cell type, or under more precise environmental conditions or developmental control. Promoters directing expression in these instances are known as inducible promoters. In the case where a tissue-specific promoter is used, protein expression is particularly high in the tissue from which extraction of the protein is desired. Depending on the desired tissue, expression may be targeted to the endosperm, aleurone layer, embryo (or its parts as scutellum and cotyledons), pericarp, stem, leaves, tubers, roots, etc. Examples of known tissue-specific promoters include the tuber-directed class I patatin promoter, the promoters associated with potato tuber ADPGPP genes, the soybean promoter of beta-conglycinin (7S protein) which drives seed-directed transcription, and seed-directed promoters such as those from the zein genes of maize endosperm and rice glutelin-1 promoter. See, e.g., Bevan et al., 14 Nucleic Acids Res. 4625-38 (1986); Muller et al., 224 Mol. Gen. Genet. 136-46 (1990); Bray, 172 Planta 364-70 (1987); Pedersen et al., 29 Cell 1015-26 (1982); Russell & Fromm, 6 Transgenic Res. 157-58 (1997).

In a preferred aspect of the invention, the cytokine of interest is produced from seed by way of seed-based production techniques using, for example, canola, corn, soybeans, rice and barley seed. See, e.g., Russell, 240 Current Technologies in Microbiol. & Immunol. 119-38 (1999). In such a process, the desired protein is recovered during or after seed maturation, or during the germination phase.

Protein purification: broadly defined, any process by which proteins are separated from other elements or compounds on the basis of charge, molecular size, or binding affinity. More specifically, the expressed recombinant cytokines of the invention may be purified to homogeneity by chromatography. In one embodiment, the cytokine produced in corn seed is purified by extraction/precipitation, followed by cation exchange column chromatography, followed by purification by anion exchange column chromatography. However, other purification techniques known in the art can also be used, including ion exchange chromatography, and reverse-phase chromatography and selective phase separation. See, e.g., Maniatis et al., Mol. Cloning: A Lab. Manual (Cold Spring Harbor Laboratory, N.Y. 1989); Ausubel et al., Current Protocols in Mol. Bio. (Greene Publishing Associates and Wiley Interscience, N.Y. 1989); Scopes, Protein Purification: Principles & Practice (Springer-Verlag New York, Inc., N.Y. 1994); U.S. Pat. Nos. 5,990,284, 5,804694, and 6,037,456.

Reading frame: refers to the preferred way (of three possible) of reading a nucleotide sequence as a series of triplets. Reading “in frame” means that the nucleotide triplets (codons) are translated into a nascent amino acid sequence of the desired recombinant cytokine. Specifically, the present invention contemplates a first nucleic acid linked in reading frame to a second nucleic acid.

Recombinant: as used herein, broadly describes various technologies whereby genes can be cloned, DNA can be sequenced, and protein products can be produced. As used herein, the term also describes proteins that have been produced following the transfer of genes into the cells of plant host systems.

Structural gene: a gene coding for a polypeptide that may be equipped with a suitable promoter, termination sequence and optionally other regulatory DNA sequences, and having a correct reading frame.

Total soluble protein: relative portion of desired measured protein compared to total extracted protein.

Transgene: an engineered gene comprising a promoter to start gene expression, a 5′ untranslated region to initiate translation, a protein coding region, and a polyadenylation/termination region to stop gene expression. An intervening sequence (intron or IVS) may be included after the promoter, to potentially enhance expression. The protein coding region may include the desired protein to be produced, and possibly a signal peptide or fusion to an additional region(s) that allows protein targeting, stabilization, and/or purification.

Transgenic: a plant host system engineered to contain a novel, laboratory designed transgene.

Transgenic plants: plant host systems that have been subjected to one or more methods of genetic transformation; plants that have been produced following the transfer of genes into the cells of plant host systems.

Variant: an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art, for example, DNASTAR© software.

Plant Expression Vectors

Expression vectors useful in the present invention comprise a nucleic acid sequence encoding a cytokine expression cassette, designed for operation in plants, with companion sequences upstream and downstream from the expression cassette. The companion sequences may be of plasmid or viral origin and provide necessary characteristics to the vector to permit the vectors to be generated in bacteria and then introduced to the desired plant host system. A cloning vector of this invention is designed so that a coding nucleic acid sequence inserted at a particular site will be transcribed and translated. A typical expression vector may contain a promoter, selection marker, nucleic acids encoding signal sequences, and regulatory sequences, e.g., polyadenylation sites, 5′-untranslated regions, and 3′-untranslated regions, termination sites, and enhancers. “Vectors” include viral derived vectors, bacterial derived vectors, plant derived vectors and insect derived vectors.

The basic bacterial/plant vector construct may preferably comprise a broad host range prokaryote replication origin; a prokaryote selectable marker; and, for Agrobacterium transformations, T-DNA sequences for Agrobacterium-mediated transfer to plant chromosomes. Where the cytokine gene is not readily amenable to detection, the construct will preferably also have a selectable marker gene suitable for determining if a plant cell has been transformed. A general review of suitable markers for the members of the grass family is found in Wilmink & Dons, 11(2) Plant Mol. Biol. Reptr. 165-85 (1993).

Sequences suitable for permitting integration of the heterologous sequences into the plant genome may be used as well. These might include transposon sequences, and the like, Cre/lox sequences and host genome fragments for homologous recombination, as well as Ti sequences which permit random insertion of a cytokine expression cassette into a plant genome.

Suitable prokaryote selectable markers, useful for preparation of plant expression cassettes, include resistance toward antibiotics such as ampicillin, tetracycline, or kanamycin. Other DNA sequences encoding additional functions may also be present in the vector, as is known in the art. Usually, the plant selectable marker gene will encode antibiotic resistance, with suitable genes including at least one set of genes coding for resistance to the antibiotic spectinomycin, the streptomycin phosphotransferase (spt) gene coding for streptomycin resistance, the neomycin phosphotransferase (nptII) gene encoding kanamycin or geneticin resistance, the hygromycin phosphotransferase (hpt or aphiv) gene encoding resistance to hygromycin, acetolactate synthase (als) genes and modifications encoding resistance to, in particular, the sulfonylurea-type herbicides, genes coding for resistance to herbicides which act to inhibit the action of glutamine synthase such as phosphinothricin or basta (e.g., the bar gene), or other similar genes known in the art.

The constructs of the subject invention will include the expression vector for expression of the cytokine of interest. Generally, there will be at least one expression cassette, and two or more are feasible, including a selection cassette. The recombinant expression vector contains, in addition to the nucleic acid sequence encoding the cytokine of interest, at least one of the following elements: a promoter region, signal sequence, 5′ untranslated sequences, initiation codon depending upon whether or not the cytokine structural gene comes equipped with one, and transcription and translation termination sequences.

In a preferred aspect of the present invention, a gene encoding the cytokine of interest is inserted into an appropriate expression vector, i.e., a vector which contains the necessary elements for the transcription and translation of the inserted coding sequence, or in the case of an RNA viral vector, the necessary elements for replication and translation. Methods for providing transgenic plants of the present invention include constructing expression vectors containing a protein coding sequence, and/or an appropriate signal peptide coding sequence, and appropriate transcriptional/translational control signals. These methods include in vitro recombinant DNA techniques, synthetic techniques and in vivo recombination/genetic recombination. See, e g., Transgenic Plants: Prod. Sys. for Indus. & Pharm. Proteins (Owen & Pen eds., John Wiley & Sons, 1996); Galun & Breiman Des, Transgenic Plants (Imperial College Press, 1997); Applied Plant BioTech. (Chopra, Malik, & Bhat eds., Sci. Pubs., Inc., 1999); U.S. Pat. Nos. 5,620,882; 5,959,177; 5,639,947; 5,202,422; 4,956,282; WO 98/10062; WO 97/38710.

Signal Sequence

Also included in chimeric genes used in the practice of the methods of the present invention are signal sequences. In addition to encoding the cytokine of interest, the chimeric gene also encodes a signal peptide that allows processing and translocation of the protein, as appropriate. The signal sequences may be derived from mammals, or from plants such as wheat, barley, cotton, rice, soy, and potato. These signal sequences will direct the cytokine of interest to a sub-cellular location (e.g., cytosol, endoplasmic reticulum, plastid, and chloroplast) within the plant host system. This may result in increased accumulation and easier purification of the cytokine of interest. The signal peptides contemplated by the present invention include the tobacco extensin signal, the ubiquitin derived from yeast and potato, and the soy oleosin oil body binding protein. U.S. Pat Nos. 5,773,705 and 5,650,554.

Those of skill can routinely identify new signal peptides. For example, plant secretory signal peptides typically have a tripartite structure, with positively-charged amino acids at the N-terminal end, followed by a hydrophobic region and then the cleavage site within a region of reduced hydrophobicity. Although sequence homology is not always present in the signal peptides, hydrophilicity plots demonstrate that the signal peptides of these genes are relatively hydrophobic. See generally, Stryer, Biochem. 768-70 (3rd ed., W.H. Freeman & Co., N.Y., 1988). The conservation of this mechanism is demonstrated by the fact that cereal α-amylase signal peptides are recognized and cleaved in foreign hosts such as E. coli and S. cerevisiae, however particular signal sequences may allow higher expression in some hosts.

The flexibility of this mechanism is reflected in the wide range of polypeptide sequences that can serve as signal peptides. Thus, the ability of a sequence to function as a signal peptide may not be evident from casual inspection of the amino acid sequence. Methods designed to predict signal peptide cleavage sites identify the correct site for only about 75% of the sequences analyzed. See Heijne, Cleavage-Site Motifs in Protein Targeting Sequences, in 14 Genetic Eng'g (Setlow ed., Plenum Press, N.Y. 1992).

Transcription and Translation Terminators

The expression vectors of the present invention typically have a transcriptional termination region at the opposite end from the transcription initiation regulatory region. The transcriptional termination region may normally be associated with the transcriptional initiation region or from a different gene. The transcriptional termination region may be selected, particularly for stability of the mRNA to enhance expression. Illustrative transcriptional termination regions include the NOS terminator from Agrobacterium Ti plasmid and the rice α-amylase terminator.

The transcription termination process also signals for the addition of polyadenylation tails added to the gene transcription product. Alber & Kawasaki, 1 Mol. & Appl. Genetics 419-34 (1982). Polyadenylation sequences include but are not limited to those defined in the Agrobacterium octopine synthetase signal, (Gielen, et al., 3 Embo J. 835-46 (1984)), or the nopaline synthase of the same species (Depicker, et al., 1 Mol. Appl. Genetics 561-73 (1982)).

Nucleic acids

In accordance with the invention, polynucleotide sequences which encode the cytokine of interest may be used to generate recombinant nucleic acid sequences that direct the expression of such proteins, or functional equivalents thereof, in plant cells.

It will be appreciated by those skilled in the art that as a result of the degeneracy of the genetic code, a multitude of polynucleotide sequences encoding the cytokine of interest some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code.

The present invention contemplates the production in plants of cytokines that have not yet been discovered. New cytokines for which nucleic acid sequences are not available may be obtained from cDNA libraries prepared from tissues believed to possess a “novel” type of cytokine at a detectable level. For example, a cDNA library could be constructed by obtaining polyadenylated mRNA from a cell line known to express the novel cytokine, or a cDNA library previously made to the tissue/cell type could be used. The cDNA library is screened with appropriate nucleic acid probes, and/or the library is screened with suitable polyclonal or monoclonal antibodies that specifically recognize other heterologous polypeptides. Appropriate nucleic acid probes include oligonucleotide probes that encode known portions of the novel cytokine from the same or different species. Other suitable probes include, without limitation, oligonucleotides, cDNAs, or fragments thereof that encode the same or similar gene, and/or homologous genomic DNAs or fragments thereof. Screening the cDNA or genomic library with the selected probe may be accomplished using standard procedures known to those in the art. See, e.g., Ch. 10-12, Sambrook et al., Mol. Cloning: A Lab. Manual (Cold Spring Harbor Lab. Press, N.Y., 1989). Other means for identifying novel cytokines may involve known techniques of recombinant DNA technology, such as by direct expression cloning or using the polymerase chain reaction (PCR). See U.S. Pat. No. 4,683,195; Ch. 14 of Sambrook, supra; Ch. 15, Current Protocols in Mol. Bio. (Ausubel et al., eds., Greene Pub. Assocs. & Wiley-Intersci. 1991).

Altered DNA sequences which may be used in accordance with the invention include deletions, additions or substitutions of different nucleotide residues resulting in a sequence that encodes the same or a functionally equivalent gene product. The gene product itself may contain deletions, additions or substitutions of amino acid residues within a cytokine sequence, which result in a functionally equivalent cytokine. Altered nucleic acid sequences include nucleic acid sequences encoding a cytokine, or functional equivalent thereof, including those sequences with deletions, insertions, or substitutions of different nucleotides resulting in a polynucleotide that encodes the same or a functionally equivalent cytokine. Included within this definition are polymorphisms which may or may not be readily detectable using a particular oligonucleotide probe of the polynucleotide encoding a cytokine and improper or unexpected hybridization to alleles, with a locus other than the normal chromosomal locus for the polynucleotide sequence encoding a cytokine. The encoded protein may also be “altered” and contain deletions, insertions, or substitutions of amino acid residues which produce a silent change and result in a functionally equivalent cytokine. Deliberate amino acid substitutions may be made on the basis of similarity in polarity, charge, solubility, hydrophobicity, hydrophilicity, and/or the amphipathic nature of the residues as long as the biological or immunological activity of the cytokine is retained. For example, negatively charged amino acids include aspartic acid and glutamic acid; positively charged amino acids include lysine and arginine; and amino acids with uncharged polar head groups having similar hydrophilicity values include leucine, isoleucine, and valine; glycine and alanine; asparagine and glutamine; serine and threonine; and phenylalanine and tyrosine.

The nucleic acid sequences of the invention may be engineered in order to alter the coding sequence for a variety of ends including, but not limited to, alterations that modify expression and processing of the gene product. For example, alternative secretory signals may be substituted for or used in addition to the native secretory signal. See, e.g., U.S. Pat. No. 5,716,802. More specifically, the KDEL sequence has been shown to increase the expression of single-chain antibody in tobacco. Schouten et al., 30(4) Plant Mol. Biol. 781-93 (1996). Additional mutations may be introduced using techniques which are well known in the art, e.g., site-directed mutagenesis, to insert new restriction sites, or alter glycosylation or phosphorylation patterns.

Additionally, when expressing in non-human cells, the polynucleotides encoding the cytokine may be modified in the silent position of any triplet amino acid codon so as to better conform to the codon preference of the particular host organism. More specifically, translational efficiency of a protein in a given host organism can be regulated through codon bias, meaning that the available 61 codons for a total of 20 amino acids are not evenly used in translation, an observation that has been made for prokaryotes (Kane, 6 Current Op. Biotech. 494-500 (1995)), and eukaryotes (Ernst, Codon Usage & Gene Expression 196-99 (Elsevier Pub., Cambridge 1988). An application of these observations, i.e., the adaptation of the codon bias of a bacterial gene to the codon bias of a higher plant, resulted in significantly higher accumulation of the foreign protein in the plant. Perlak et al., 88(8) P.N.A.S. 3324-28 (1991); see also Murray et al., 17 Nucl. Acids Res. 477-98 (1989); U.S. Pat. No. 6,121,014. Codon usage tables have been established not only for organisms, but also for organelles and specific tissues (Kazusa DNA Research Inst., <www.kazusa.or.jp>), and their general availability enables researchers to adopt the codon usage of a given gene to the host organism. Other factors like the context of the initiator methionine start codon (Kozak, 234 Gene 187-208 (1999)), may influence the translation rate of a given protein in a host organism, and can therefore be taken into consideration. See also Taylor et al., 210 Mol. Genetics 572-77 (1987). Translation may also be optimized by reference to codon sequences that may generate potential signals of intron splice sites. Plant Mol. Bio. Labfax (Croy, ed. 1993), mRNA instability and polyadenylation signals (Perlak et al., supra).

The nucleic acid sequences of the invention are further directed to sequences that encode variants of the described cytokine. These amino acid sequence variants of a cytokine may be prepared by methods known in the art by introducing appropriate nucleotide changes into an authentic or variant cytokine encoding polynucleotide. There are two variables in the construction of amino acid sequence variants: the location of the mutation and the nature of the mutation. The amino acid sequence variants are preferably constructed by mutating the polynucleotide to give an amino acid sequence that does not occur in nature. These amino acid alterations can be made at sites that differ in cytokines, from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site.

Amino acids are divided into groups based on the properties of their side chains (polarity, charge, solubility, hydrophobicity, hydrophilicity, and/or the amphipathic nature): (1) hydrophobic (leu, met, ala, ile); (2) neutral hydrophobic (cys, ser, thr); (3) acidic (asp, glu); (4) weakly basic (asn, gln, his); (5) strongly basic (lys, arg); (6) residues that influence chain orientation (gly, pro); and (7) aromatic (trp, tyr, phe). Conservative changes encompass variants of an amino acid position that are within the same group as the native amino acid. Moderately conservative changes encompass variants of an amino acid position that are in a group that is closely related to the native amino acid (e.g., neutral hydrophobic to weakly basic). Non-conservative changes encompass variants of an amino acid position that are in a group that is distantly related to the “native” amino acid (e.g., hydrophobic to strongly basic or acidic).

Amino acid sequence deletions generally may range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells.

In one method, polynucleotides encoding a cytokine are changed via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art and this technique is exemplified by publications such as, Adelman et al., 2 DNA 183-93 (1983). A versatile and efficient method for producing site-specific changes in a polynucleotide sequence was published by Zoller & Smith, 10 Nucleic Acids Res. 6487-500 (1982).

Mutations provide one or more unique restriction sites and do not alter the amino acid sequence encoded by the nucleic acid molecule, but merely provide unique restriction sites useful for manipulation of the molecule. Thus, the modified molecule would be made up of a number of discrete regions, or D-regions, flanked by unique restriction sites. These discrete regions of the molecule are herein referred to as cassettes. Molecules formed of multiple copies of a cassette are another variant of the present gene which is encompassed by the present invention. Recombinant or mutant nucleic acid molecules or cassettes which provide desired characteristics such as resistance to endogenous enzymes such as collagenase are also encompassed by the present invention.

PCR may also be used to create amino acid sequence variants of a recombinant cytokine. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the cytokine at the position specified by the primer. The product DNA fragments replace the corresponding region in the plasmid and this gives the desired amino acid variant.

A further technique for generating amino acid variants is the cassette mutagenesis technique described in Wells et al., 34 Gene 315 (1985); and other mutagenesis techniques well known in the art, such as, for example, the techniques in Sambrook et al., supra; Ausubel et al., Current Protocols in Mol. Biol. supra.

Due to the inherent degeneracy of the genetic code, other DNA sequences which encode substantially the same or a functionally equivalent amino acid sequence or polypeptide, specifically, comprising a consistent (Gly-X-Y), amino acid structure, that are natural, synthetic, semi-synthetic, or -recombinant, may be used in the practice of the claimed invention. Such DNA sequences may be include those which are capable of hybridizing to the appropriate cytokine sequence under stringent conditions.

Thus, the invention further relates to nucleic acid sequences that hybridize to the above-described sequences. In particular, the invention relates to nucleic acid sequences that hybridize under stringent conditions to the above-described nucleic acids. As used herein, the terms “stringent conditions” and “stringent hybridization conditions” mean that hybridization will generally occur if there is at least 95% and preferably at least 97% identity between the sequences. An example of stringent hybridization conditions is overnight incubation at 42° C. in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 micrograms/milliliter denatured, sheared salmon sperm DNA, followed by washing the hybridization support in 0.1×SSC at approximately 65° C. Other hybridization and wash conditions are well known and are exemplified in Sambrook, et al., Molecular Cloning: A Laboratory Manual (2d ed., Cold Spring Harbor, N.Y. (1989)), particularly Chapter 11.

Transformation of Plant Cells

Transformation is a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, A. tumefaciens-mediated transfection, and particle bombardment.

More specifically, standard methods for the transformation of rice, wheat, corn, sorghum, and barley are described in the art. See Christou et al., 10 Trends in Biotech. 239 (1992); Lee et al., 88 P.N.A.S. 6389-93 (1991). Wheat can be transformed by techniques similar to those employed for transforming corn or rice. Furthermore, Casas et al., 90 P.N.A.S. 11212-16 (1993), describe a method for transforming sorghum, while Lazzeri, 49 Methods Mol. Biol. 95-106 (1995), teach a method for transforming barley. Suitable methods for corn transformation are provided by Fromm et al., 8 Bio/Technology 833-39 (1990); Gordon-Kamm et al., 2 Plant Cell 603-18 (1990); Russell et al., 6 Transgenic Res., 157-58 (1997); U.S. Pat. No. 5,780,708.

Vectors useful in the practice of the present invention may be microinjected directly into plant cells by use of micropipettes to mechanically transfer the recombinant DNA. Crossway, 202 Mol. Gen. Genet., 179-85 (1985). The genetic material may also be transferred into the plant cell by using polyethylene glycol, Krens et al., 96 Nature 72-74 (1982).

Another method of introduction of nucleic acid segments is high velocity ballistic penetration by small particles with the nucleic acid either within the matrix of small beads or particles, or on the surface. Klein et al., 327 Nature 70-73 (1987); Knudsen & Muller, 185 Planta 330-36 (1991).

Additionally, another method of introduction would be fusion of protoplasts with other entities, either minicells, cells, lysosomes or other fusible lipid-surfaced bodies, Fraley et al., 79 P.N.A.S. 1859-63 (1982).

The vector may also be introduced into the plant cells by electroporation. (Fromm et al., 82 P.N.A.S. 5824-28 (1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus. See U.S. Pat. No. 5,584,807.

Isolating Progeny Containing Cytokine of Interest

Progeny containing the desired cytokine can be identified by assaying for the presence of the biologically active heterologous protein using assay methods well known in the art. Such methods include Western blotting, immunoassays, binding assays, and any assay designed to detect a biologically functional heterologous protein. See, for example, the assays described in Klein, Immunology: Sci of Self-Nonself Discrimination (John Wiley & Sons eds., New York, N.Y. 1982).

Preferred screening assays detect the biological activity of the cytokine. These assays identify, for example, the production of a complex, formation of a catalytic reaction product, the release or uptake of energy, cell growth, identification as authentic by the appropriate antibody, and the like. For example, a progeny containing a cytokine molecule produced by this method may be recognized by an antibody to binds to an authentic antigenic site on the cytokine in a standard immunoassay such as an ELISA or other immunoassays known in the art. See Antibodies: A Lab. Manual (Harlow & Lane, eds., Cold Spring Harbor Laboratory, Cold Spring Harbor, N.Y. 1988).

Plant Regeneration

After determination of the presence and expression of the desired gene products, whole plant regeneration is desired. Plant regeneration from cultured protoplasts is described in Evans, et al., Handbook of Plant Cell Cultures, Vol. 1: (MacMillan Publishing Co. New York 1983); Cell Culture & Somatic Cell Genetics of Plants, (Vasil I. R., ed., Acad. Press, Orlando, Vol. I 1984, and Vol. III 1986).

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables, dicots, and monocots.

Methods for regeneration vary from species to species of plants, but generally a cell capable of being cultured either alone or as part of a tissue and containing copies of the cytokine gene is first provided. Callus tissue may be formed and shoots may be induced from callus and subsequently rooted, or shoots may be induced directly from a cell within a meristem.

Alternatively, embryo formation can be induced from the cell suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

A plant of the present invention containing the expression vector comprised of a first nucleic acid sequence that is capable of regulating the transcription of a second nucleic acid sequence encoding a significant portion of a peptide that is capable of targeting a protein to a sub-cellular location and fused to this second nucleic acid, a third nucleic acid encoding the cytokine of interest, is cultivated using methods well known to one skilled in the art. Any of the transgenic plants of the present invention may be cultivated to isolate the desired cytokine they contain.

After cultivation, the transgenic plant is harvested to recover the produced cytokine. This harvesting step may consist of harvesting the entire plant, or only the leaves, or roots of the plant. This step may either kill the plant or if only the portion of the transgenic plant is harvested may allow the remainder of the plant to continue to grow.

The transgenic plants according to this invention can be also be used to develop hybrids or novel varieties embodying the desired traits. Such plants would be developed using traditional selection type breeding.

The mature plants, grown from the transformed plant cells, are selfed and non-segregating, and the resulting homozygous transgenic plants is identified. Alternatively, an outcross can be performed, to move the gene into another plant. In either case, the transgenic plants produces seed containing the proteins of the present invention. The transgenic plants according to this invention can be used to develop hybrids or novel varieties embodying the desired traits. Such plants would be developed using traditional selection type breeding.

The following examples will illustrate the invention in greater detail, although it will be understood that the invention is not limited to these specific examples. Various other examples will be apparent to the person skilled in the art after reading the present disclosure without departing from the spirit and scope of the invention. It is intended that all such other examples be included within the scope of the appended claims.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever. The following techniques can be adapted by one skilled in the art to produce, in any appropriate plant host system, a cytokine of interest.

### Example 1

Construction of a Vector for Expression of hGH in Corn Seeds

The initial plant expression vector (accepting vector) used contained the CaMV 35S promoter (P-35S), a plant-active 5′utr and signal peptide with an NcoI site for fusion to the start methionine of the hGH sequence, and a 3′utr/polyA addition site (nos). This combination has been used to express a single chain antibody in plant cells (Francisco et al., 1997). The signal peptide for directing the protein through the secretory path is a 26 amino acid version from Nicotiana plumbaginifolia. De Loose et al., 99 Gene 95-100 (1991).

The plant cell expression cassette containing the hGH gene (GenBank accession number AF205361) was derived from an expression cassette originally designed for direct expression in E. coli. Staub et al., 18 Nat. Biotech. 333-38 (2000). The E. coli cassette contains methionine and alanine codons, in the context of an NcoI site immediately upstream from the codons encoding the authentic mature amino terminus (beginning Phe-Pro-Thr) of native hGH. The downstream end of the coding sequence used a HindIII restriction site after the stop codon. This hGH cassette was put into the NcoI-PstI site of the above accepting vector, by using a linker: dar100: (agcttgca) to allow joining of the HindIII and PstI sites, and to regenerate the HindIII site. The resulting plasmid was called pwrg4738.

Modifications were made in pwrg4738 for ease of handling, and to design the encoded hGH with proper amino terminus. First, the SacI site downstream of the nos was eliminated by cutting pwrg4738 with KpnI and EcoRI, and ligating the vector fragment with the linker: dar73: (aattgtac).

Next, the region between the BlpI site in the signal peptide and the now unique SacI site in the hGH was replaced with a complementary oligo that eliminated the extra Met and Ala codons at the beginning of hGH. The resulting plasmid was called pwrg 4776. The oligomers used, dar139 (kinased) and dar140, are shown below: dar139:

ttagctagcgaaagctccgccttcccgactatcccactgagccgcctgttcgacaacgctatgctgcgagct (SEQ ID NO:01) dar140:

cgcagcatagcgttgtcgaacaggcggctcagtgggatagtcgggaaggcggagctttcgctagc (SEQ ID NO:02)

The corn transformation vector was designed to include a corn seed endosperm expression cassette, and a corn selectable marker cassette. The corn seed endosperm expression cassette includes an endosperm-specific promoter from rice (P-OsGT1) that has been used in corn seed previously (Russell & Fromm, 6 Transgenic Res. 157-68 (1997); WO 98/10062), a corn HSP70 intron (IVS) (WO 93/19189), a polyadenylation region previously used in corn (nos) (WO 98/10062). The corn selectable marker cassette includes the 35S promoter, neomycin phosphotransferase II coding region (NPT2), and a polyadenylation region (nos).

The construction of the corn transformation vector used the HindIII to BlpI fragment of pwrg4768, encompassing the 5′utr, IVS, and amino terminus of the signal peptide. A second fragment came from pwrg4776, extending from BlpI to XbaI, encompassing the carboxy-terminus of the signal peptide, the entire hGH coding region, and nos polyadenylation region. These fragments were ligated into the corn transformation vector pwrg4789, having a HindIII site directly after the seed promoter, and an XbaI site directly before the selection cassette. The resulting plasmid, pwrg4825, is illustrated in FIG. 2. General methods for constructing plant expression vectors have been described. See, e.g., Staub et al., 2000).

### Example 2

hGH Transient Expression with Intracellular Targeting

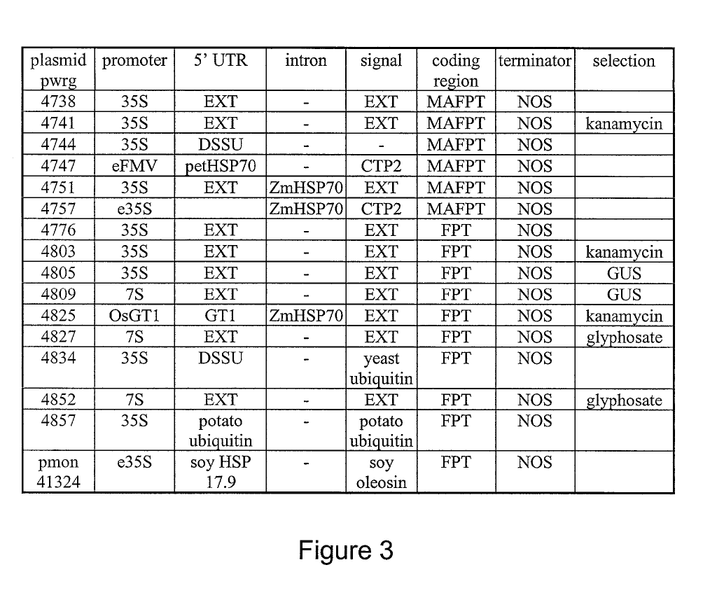

Transient expression, achieved using constitutive promoters, allows examination of gene expression and protein accumulation in multiple plant tissues and species. Gene construct can be tested quickly for gross quality and quantity performance, although details of protein quality (N-terminus, glycosylation) may require transgenic plants. The list of vectors encoding hGH for transient expression in several plant cells types is illustrated in FIG. 3. The 35S, extensin, nos, and kanamycin selection elements have been described. Russell et al., U.S. Pat. No. 6,140,075; Francisco et al. 8 Bioconjugate Chem. 708-13 (1997). The ZmHSP70 intron is described in Brown et al., U.S. Pat. No. 5,859,347. The Petunia HSP70 5′ UTR is described in Austin et al., U.S. Pat. No. 5,659,122. The rice glutellin promoter (OsGT1) for monocot seed expression is described in Brar et al. WO/9810062. The bean 7S promoter for dicot seed expression is described in Chen et al., 83 P.N.A.S 8560-64. The FMV promoter is described in Rogers, U.S. Pat. No. 6,018,100. The DSSU 5′ UTR and GUS selection cassette used for soy transformation is described in Kridl, WO/0009721. The CTP2 and glyphosate selection cassette is described in Barry et al., U.S. Pat. No. 5,633,435. The potato ubiquitin 3 used for fusion to hGH is described in Garbarino et al. 24 Plant Mol. Biol. 119-27 (1994).

Three different expression vectors were constructed for transiently expressing and targeting hGH to different locations within the plant cell. These expression vectors included an hGH expression cassette employing the CaMV 35S promoter, a plant active 3′UTR/nos polyA, and different plant-active 5′ regulatory regions. The differing 5′ regulatory regions that targeted the expressed hGH to different locations within the plant cell as follows: (1) a 5′ regulatory region that targeted hGH to the cytosol (“cytosolic form”); (2) a 5′ regulatory region that that targeted hGH to the endoplasmic reticulum (“secreted form”); and (3) a chloroplast transit peptide 5′ regulatory region that targeted hGH to the plastid (“plastid form”).

The hGH gene cassette used in the three expression vectors was designed originally for the direct translation and expression of the hGH protein in E. coli. In this vector, the hGH cassette contained a Nco I restriction site at the N-terminal region, and yielded a methionine then an alanine codon immediately preceding the natural PheProThr N-terminus of mature hGH.

The first expression vector, targeting the cytosol, included the hGH structural gene, the CaMV 35S promoter, a plant-active 5′UTR, and a 3′UTR/Nos poly A signal. This generated a methionine-alanine N-terminus on the expressed hGH, which is not identical to the natural hGH N-terminus (PheProThr).

The second expression vector, targeting the secretory pathway, included the hGH structural gene, a 5 ′ regulatory region encoding a signal peptide to facilitate secretion of the nascent protein through the endoplasmic reticulum, and a 3 ′UTR/nos poly A signal. This expression vector also comprised the AlaSerAla/MetAlaPhe (SEQ ID NO:03) fusion point between the signal peptide and N-terminus of hGH and generated the methionine N-terminus on the expressed hGH protein. This expression vector was further modified by introducing an intron from the corn heat shock 70 gene between the promoter and the signal peptide.

The third expression vector, targeting the plastid, comprised the hGH structural gene fused to the CaMV 35S promoter, a 5′ regulatory region that encoding a plastid targeting sequence, and a 3′UTR/nos poly A addition signal. This expression vector was further modified by introducing an intron from the corn heat shock 70 gene between the promoter and the signal peptide. This expression vector also contained an CysMetLeuAla/MetAlaPhe (SEQ ID NO:04) fusion point, that also generated a methionine N-terminus on the expressed hGH.

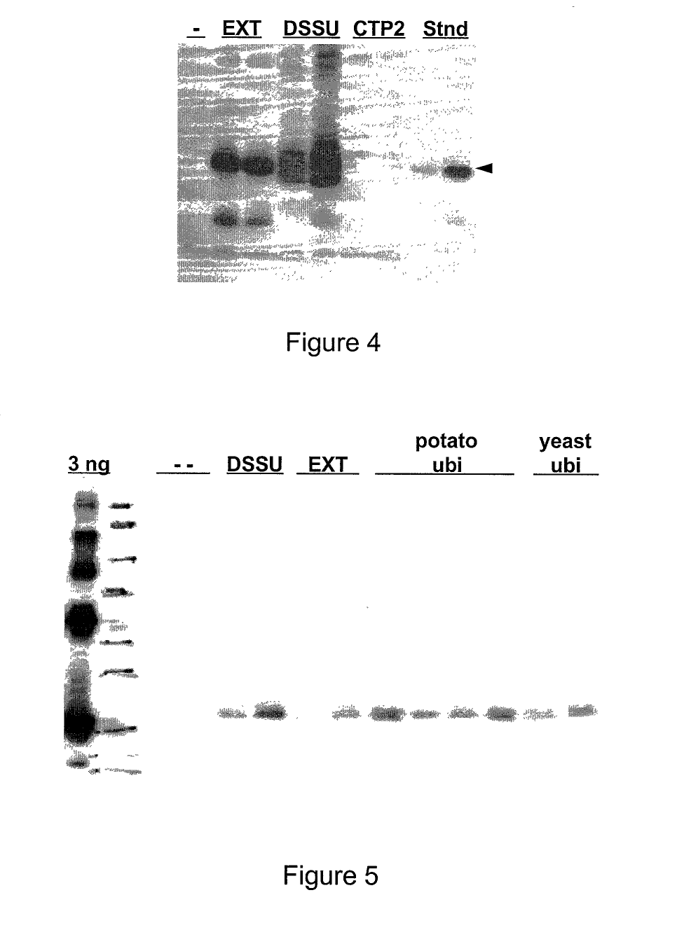

These three expression cassettes were first expanded in E. coli from which the DNAs were then purified. Next, the plasmid DNA was coated onto gold beads is transformed into soybean embryos by particle bombardment as described in U.S. Pat. No. 5,914,451. More specifically, soy embryo hypocotyl target tissue is prepared by overnight germination of soy seeds. After gene delivery and 30-50 hr of incubation on nutrient media, the entire leaf section or the treated surface of the hypocotyls is isolated, ground in PBS, clarified by centrifugation, and the extract separated by reducing polyacrylamide gel electrophoresis (reducing PAGE). The separated proteins are transferred to nitrocellulose or PVDF membrane. The blot is analyzed via Western blot by reaction with rabbit-anti-hGH (Biodesign International D710071R), followed by detection with horse radish peroxidase-conjugated goat-anti-rabbit antibody (Sigma A0545) and substrate (ECL; Amersham). FIG. 4 shows the result for soy hypocotyls. A comparison of the constructs indicated very low hGH expression with the plastid targeting signal (CTP2), higher hGH expression levels with the construct containing the secretion signal (EXT), and the highest hGH expression levels with the cytosolic construct (DSSU). Additionally, there was also a 14 kD truncation product associated with the secreted form. There was also a truncation product associated with the cytosolic form, but this was less prevalent in comparison to the secreted form.

The high level of hGH expression with the cytosolic construct was an unexpected, but otherwise desired result. The advantages of the having high hGH expression levels with the cytosolic form include a reduced cost in production and easier purification.

Ubiquitin Fusion Expression Constructs

Although the previously described cytosolic form of hGH had the highest level of expression, it was also expected to have the non-native MetAla N-terminus, based upon the construct design. In order to eliminate the undesirable N-terminus, two new expression constructs were designed in which the natural N-terminus of hGH was fused to ubiquitin, yielding a fusion point of LeuArgGlyGly/PheProThr (SEQ ID NO:05). This fusion point generates the desired, non-methionine N-terminus, due to the natural processing system in the plant. The protein would not be expected to pass through the secretory pathway, since it has no secretory signal.

To produce the first new construct, a yeast ubiquitin monomer was placed between the end of the DSSU 5′ UTR and the translational start of hGH. This construct was named pwrg4834. The second construct was generated by replacing the 5′UTR, signal sequence, and fragment of hGH from pwrg4776 with a splicing PCR product that included the 5′ UTR and ubiquitin monomer of potato ubiquitin gene 3, and a replacement fragment of hGH. This construct was named pwrg4857. These two new constructs were transformed into soy hypocotyls as described above. Reduced Western blot analysis (FIG. 5) from transient soy hypocotyl expression showed significant hGH expression from the cytosolic (DSSU), secreted (EXT), or cytosolic ubiquitin fusions (potato ubi, yeast ubi). The ubiquitin fusions also showed a similar mobility to the other versions, presumably because the endogenous ubiquitin processing system accurately cleaved the fusion, leaving the desired amino terminus of hGH.

Plant Oil Body-Binding Protein Fusion Expression Constructs

To eliminate the 14 kD truncation product associated with the secreted form of hGH, described above, a new vector was constructed utilizing the soy oleosin oil body-binding protein signal peptide. Oil body-binding protein has been shown to result in correct protein folding of some fused proteins normally destined for secretion, and ease protein purification from other host cell components. See, e.g., U.S. Pat. No. 5,650,554. This fusion protects the hGH from the apparent proteases in the secretory path that cleave hGH, thus yielding more, folded, intact hGH.

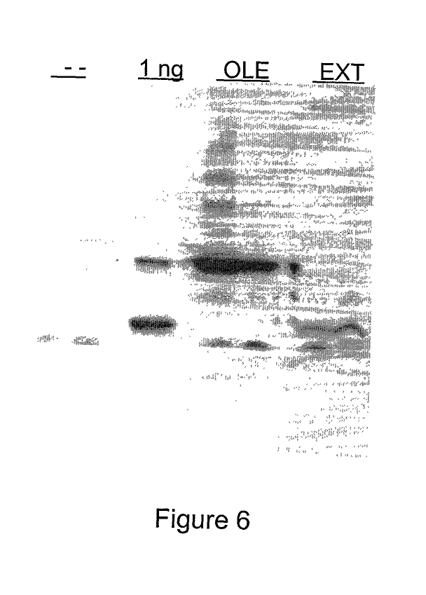

The design entailed a synthetic gene that encoded soy oleosin, an enterokinase protease recognition site, and a fragment of the hGH amino terminus. This was inserted between a plant 5′ UTR, and the remaining fragment of hGH, to create pmon41324. While the oleosin fusion may aid in correct folding and potential purification of hGH, the enterokinase site allows later specific protease cleavage at AspAspAspAspLys/PheProThr (SEQ ID NO:06), to yield the mature natural amino terminus of hGH. Reduced SDS-PAGE Western blot analysis of the transient soy hypocotyl extracts (FIG. 6) shows a significant increase in expression level of the correct-sized fusion product (OLE) relative to the non-fused extensin control (EXT), with very little evidence of the 14 kD truncated fragment. In FIG. 6, the left lane in each pair was from extractions with 20 mM Tris-Cl pH 7.5, 0.01% Triton X-100, 5% glycerol, and 50 mM NaCl. The right lane in each pair was from extractions with 20 mM Tris-Cl pH 7.5, 4 mM CHAPS, 5% glycerol, and 50 mM NaCl. The 1 ng hGH standard has a monomer band that co-migrated with the secreted hGH design, while the oleosin fusion migrated more slowly, as expected for a fusion.

### Example 3

Expression of hGH in Soy Plant with Secretory Targeting

Expression cassettes comprising the hGH structural gene operably linked to the plant extensin signal peptide, either the CAMV 35S or 7S seed storage protein promoter, and the nos poly A termination site, were used to generate transgenic soy plants. The expression cassettes were transformed into soy by particle bombardment. All designs used the hGH gene cassette as in pwrg4776, having the desired PheProThr N-terminus. It was incorporated with a β-glucuronidase expression cassette, used for selecting transformed plants. Biolistic-based plant transformation was performed essentially as described by McCabe et al., 6 Bio Tech. 923-26 (1988). An alternative gene design used a promoter from the soy 7S seed storage protein. Chen et al. 83 P.N.A.S. 8560-64 (1986). An alternative design used selection by glyphosate, using the CP4 selection cassette encoding a modified bacterial EPSPS. WO 99/51759. Another design used the same two cassettes, but in a Agrobacterium-based transformation vector. WO 00/42207. Plants were screened by the ELISA and Western methods as above.

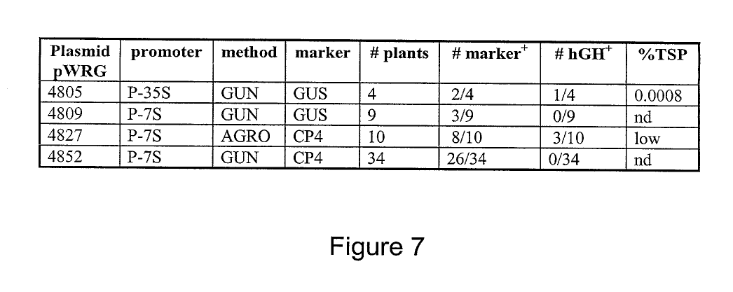

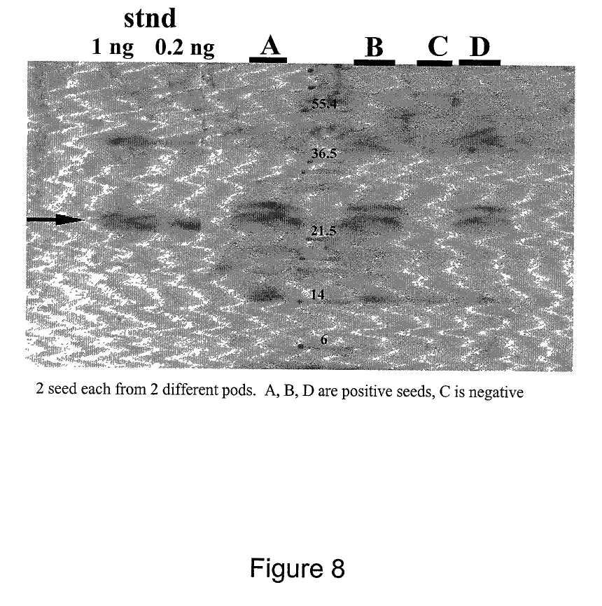

All plants showed expression in both leaves (for 35S vectors) and seeds (for all vectors). Additionally, seed expression by ELISA diminished to <0.0008% of total soluble protein upon maturity, as shown in the FIG. 7. Some of the material was of the expected molecular weight, as judged by reduced SDS-PAGE (loaded at approximately 100 μg total extracted protein from dry seeds), and Western blot of developing seeds. FIG. 8.

### Example 4

hGH Stable Cell Expression with Secretory Targeting in Stable Tobacco Cell Lines

The expression constructs described in Example 2 were also used to generate stable transgenic tobacco cell lines. These expression constructs included the cytosolic targeting expression vector, the secreted targeting expression vector, and the plastid targeting expression vector.

These expression constructs were transformed into tobacco cells by accelerated particle delivery as follows. Tobacco NT1 cells were grown in suspension culture according to the procedure described in Russell et al., 12P In Vitro Cell. Dev. Biol. 97-105 (1992), and An, 79 Plant Physiol. 568-70 (1985). Prior to bombardment, fresh tobacco suspension media (TSM) was inoculated using NT1 cells in suspension culture, and the culture was allowed to grow four days to early log phase. TSM contains, per liter, 4.31 g of M.S. salts, 5.0 ml of WPM vitamins, 30 g of sucrose, 0.2 mg of 2,4-D (dissolved in KOH before adding). The medium is adjusted to pH 5.8 prior to autoclaving. Early log phase cells were plated onto 15 mm target disks on tobacco culture medium (TCM) containing 0.3M osmoticum and held for one hour prior to bombardment. The solid medium TCM consists of TSM plus 1.6 g/1 Gelrite (Scott Labs., West Warwick, R.I.). The DNA construct was delivered into the plated NT1 cells using a spark discharge particle acceleration device as described in U.S. Pat. No. 5,120,657. Delivery voltages ranged from 12-14 kV.

Following transformation of the NT1 cells, the disks containing the cells were held in the dark for one day, during which the disks were transferred twice, at regular intervals, to solid media containing progressively lower concentrations of osmoticum. The cells were then transferred to TCM containing 350 mg kanamycin sulphate/liter and grown for 3-12 weeks, with weekly transfers to fresh media. After 3-6 weeks of growth on solid medium, kanamycin resistant calli of transgenic NT1 cells may be used to start a suspension culture in TSM containing 350 mg kanamycin sulphate/liter.

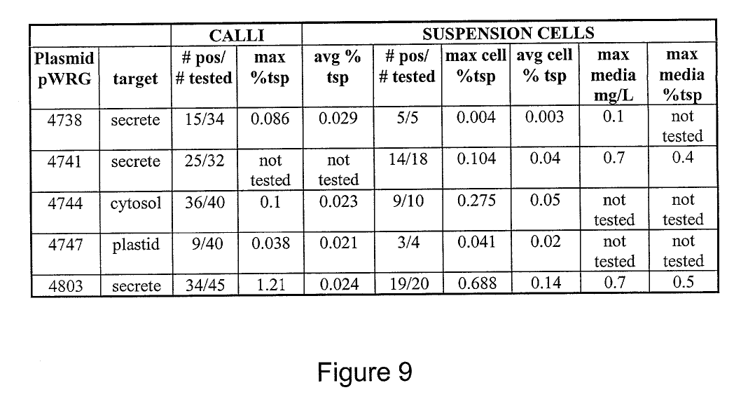

Expression of the hGH constructs in transgenic calli and suspension cells was evaluated by hGH ELISA kit (Boehringer Mannheim, Indianapolis, Ind.). The appropriate colonies were then advanced to liquid suspension culture and retested for hGH accumulation in the cells and media, as summarized in FIG. 9. Plasmid pWRG4738 was co-bombarded with a vector containing the kanaycin selection cassette, while the others had both gene cassettes on a single plasmid. Plasmid pWRG4803 was designed to have the desired PheProThr N-terminus. The ELISA results indicated a co-expression frequency (# pos/# tested) maximal expression (% max tsp), and average expression (avg % tsp) for the different targeting systems was lowest with plastid targeting, and similar with cytosolic or secreted (ER). This is similar to results seen with the transients. The plasmids designed for secretion showed maximum % tsp levels after 7 days in suspension can be higher in the media than in the cells. Higher % tsp levels can aid in purification.

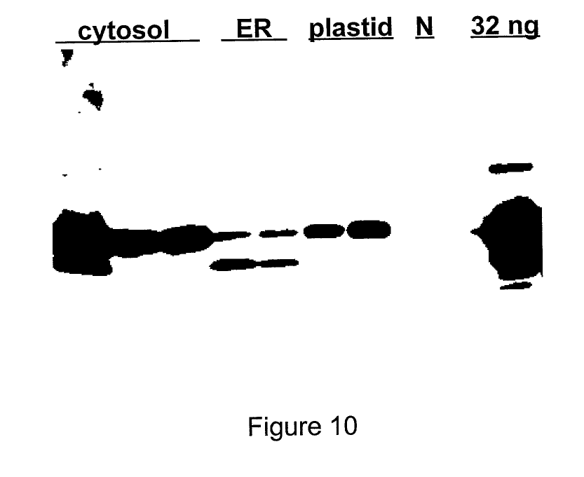

Next, transgenic calli and suspension cells were analyzed for the expression of the various forms of hGH by Western blotting with a rabbit-anti-hGH specific antibody. The results showed higher levels of the 14 kD truncation band in the secreted version than in the cytosolic and plastid expression versions. FIG. 10. The absence of the 14 kD truncation product, with the cytosolic expression cassette, is a preferred result.

### Example 5

hGH Expression with Secretory Targeting in Tobacco Plants

The expression constructs as described in Example 2 were also used to generate stable transgenic tobacco plants. These expression constructs included the cytosolic targeting expression vector, the secreted targeting expression vector, and the plastid targeting expression vector. These expression constructs were mixed with a glyphosate selection cassette, and transformed into tobacco cells by accelerated particle delivery, as set forth previously.

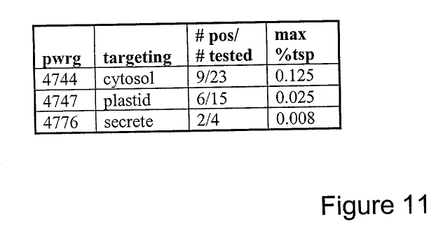

Expression of the genetic constructs in transgenic tobacco plant leaves were evaluated by Western blot with a rabbit-anti-hGH specific antibody. FIG. 11 shows the expression summary from with the different targeting of hGH. The results, which are consistent with the results of Example 4, show best expression from the cytosol-directed design. Testing more events of the secreted design may have identified higher expressers.

### Example 6

Plant Cell hGH Purification and Quality Tests MetAla-hGH Purification and Quality Test

MetAla-hGH was purified from the media of tobacco cell lines expressing the secreted version of the protein, designed to have a MetAla N-terminus. Media was collected at 4-5 days post innoculation, the pH was adjusted to 8.3 with 1M Tris base, and loaded onto a Pharmacia Biotech DE fastflow sepharose column (Pharmacia, Peapack, N.J.). Next, the column was washed with 25 mM Tris pH 8.3 and then developed with a gradient to 25 mM Tris pH 8/500 mM NaCl. The major fractions were pooled and assayed for total soluble protein and the presence of hGH. The Pierce Coomassie Plus assay (Pierce Chems., Rockford, Ill.) showed that the pooled major fractions contained 120.5 ng/ml total soluble protein. The presence of MetAla-hGH in the pooled major fractions was analyzed by ELISA using an anti-hGH antibody. The ELISA results indicated an average of 10.2 ng/μl MetAla-hGH in the pooled major fractions, indicating a purity of 8.5%.

The pooled major fractions were applied to a reducing 4-20% gradient SDS-PAGE, and then the SDS/PAGE-separated proteins were transferred onto a polyvinylidene difluoride (PVFD) membrane (Schleicher & Schuell, Inc., Keene, N.H.). The blots were stained with 0.1% Ponceau S (Sigma, St. Louis, Mo.) in 1% acetic acid, and de-stained in water. The band at the position corresponding to the appropriate size for hGH was marked and then sequenced on an Applied Biosystems sequencer (Applied Biosystems, Foster City, Calif.). Sequencing of MetAla-hGH yielded not only the expected MetAlaPhePro sequence, but also the nature-identical N-terminus of PheProThr as a minor product.

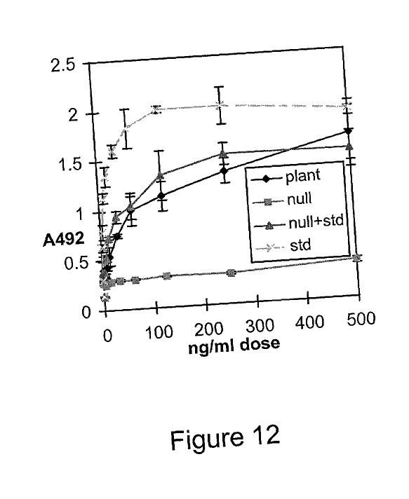

Activity tests of the partially purified MetAla-hGH were performed by the method of Dattani et al., 270 J. Biol. Chem. 9222-26 (1995), as shown in FIG. 12. Mammalian rat lymphoma Nb2 cells, which respond to hGH, were incubated with different levels of purified MetAla-hGH. Following incubation, the mammalian cells were assayed for mitotic activity and cell proliferation by the proportional conversion of tetrazolium dye to colored formazan product. (Promega, Madison, Wis.). The results indicated that the cells exhibited a dose-dependent stimulation that was above background activity. Dose response of control standard in null tobacco cell suspension media was similar to that produced by the transgenic cells, though the standard in buffer alone had a stronger response.

Phe-hGH Purification and Quality Test

Phe-hGH was purified from the media of the cell line expressing the secreted version of hGH, with the desired N-terminus. Media was collected at 4-5 days post innoculation and loaded onto a Pharmacia DEAE Streamline column (Pharmacia, Peapack, N.J.). The column was washed with 25 mM Tris pH 8.3, followed by a step elution. Coomassie staining, as described above, revealed that the pooled major fractions contained an average of 272-293 μg/ml total protein. ELISA using an anti-hGH antibody revealed that the pooled major fractions contained an average of 5.4-10.1 ng/μl Phe-hGH.

The pooled major fractions were then diluted, adjusted to pH 9.5 with Tris base, and loaded onto to a SOURCE 30 Q column. The SOURCE 30 Q column was developed with a linear gradient of 0-1 M NaCl.

The pooled major fractions were next applied to a reducing 4-20% gradient SDS-PAGE, and the SDS/PAGE-separated proteins were then transferred onto a polyvinylidene difluoride (PVFD) membrane (Schleicher & Schuell, Inc., Keene, N.H.). The blots were stained with 0.1% Ponceau S (Sigma, St. Louis, Mo.) in 1% acetic acid, then destained in a water. The band at the position corresponding to the appropriate size for hGH was marked and then sequenced on an Applied Biosystems sequencer (Applied Biosystems, Foster City, Calif.). The sequencing results revealed the preferred result of only the nature-identical N-terminus, PheProThrIlePro, being present without the presence of any hydroxyproline.

Mass Spectrophotometry of Phe-hGH

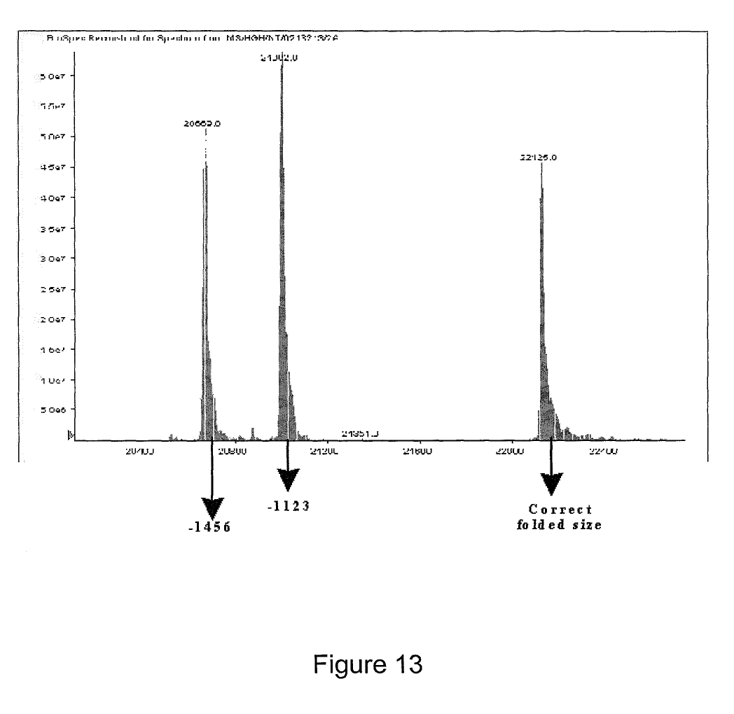

The pooled major fractions of Phe-hGH were also analyzed by mass spectrometry. The mass spectrometry results in FIG. 13 show significant levels of authentic-sized hGH at 21,255 mass units, having the proper disulfide linkages, free of novel glycosylation and amino acid modifications.

### Example 7

hGH Expression in Corn with Secretory Targeting

The corn transformation vector included an endosperm-specific expression cassette, and a corn selectable marker cassette as described in Example 1. The endosperm-specific promoter, obtained originally from rice (P-OsGT1) has been used previously in corn seed. Russell & Fromm, 6 Transgenic Research 157-68 (1997); WO 98/10062). The construct also included a corn HSP70 intron (IVS) (WO 93/19189) and a nos polyadenylation region used previously in corn (WO 98/10062). The corn selectable marker cassette included the 35S promoter, neomycin phosphotransferase II coding region (NPT2), and a polyadenylation region (nos).

The construction of the corn transformation vector used the HindIII to BlpI fragment of pwrg4768, encompassing the 5′UTR, IVS, and amino terminus of the signal peptide. A second fragment came from pwrg4776, extending from BlpI to XbaI, encompassing the carboxy-terminus of the signal peptide, the entire hGH coding region, and nos polyadenylation region. These fragments were ligated into the corn transformation vector pwrg4789, having a HindIII site directly after the seed promoter, and an XbaI site directly before the selection cassette. The resulting plasmid, pwrg4825, is illustrated in FIG. 2. General methods for constructing plasmid vectors have been described. Ausabel et al., 1999.

Corn transformation was performed by the biolistic method, using a kanamycin selection gene. Prior to use, the plasmid vector was cut with restriction enzyme NotI, cutting at sites on either side of the plant transgene cassettes. The transgene fragment was purified, eliminating the bacterial vector sequences. The transgene DNA can be precipitated onto microscopic metal particles, and delivered to corn cell material that is competent to be regenerated into a fertile corn plant. Gordon-Kamm et al., 2 Plant Cell 603-18 (1990). The corn material is then exposed to kanamycin, killing any cells that do not express the NPT2 transgene. The surviving cells are put into a series of media conditions of varied salts and plant growth regulators, stimulating the organized production of plant roots and shoots. The plantlets are then put to soil, and plants grown in the greenhouse to maturity, pollinated, and the resulting seed harvested. This seed can be either processed to purify the hGH, or replanted. Replanted mature plants can be either “selfed,” generating a pure-breeding transgenic strain, or out-crossed, placing the transgene in a novel genetic background, or used to create more transgenic material by transferring the transgenic pollen to multiple non-transgenic ears.

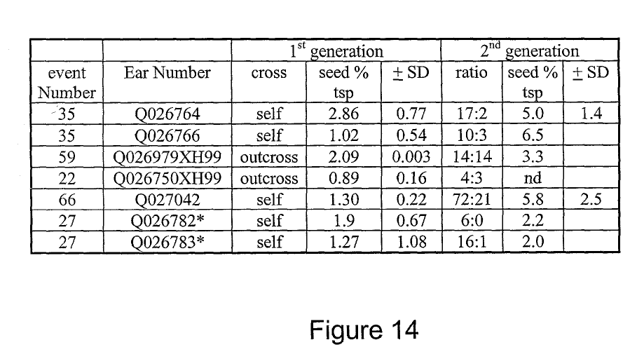

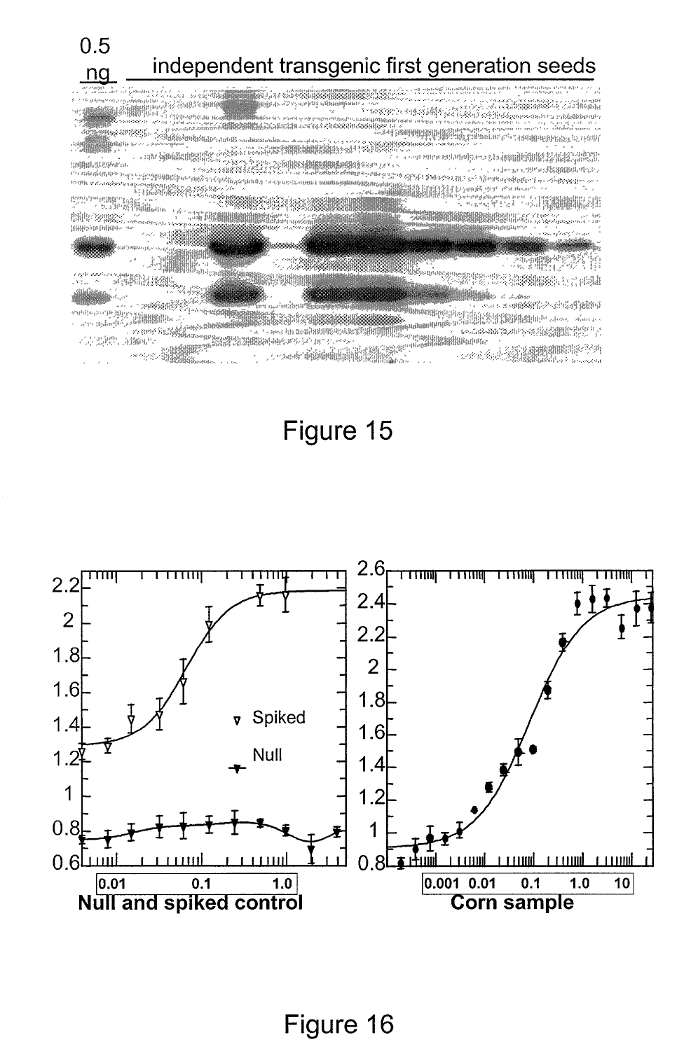

To test for expression hGH in the transgenic corn kernels, mature seeds were pulverized either individually or as a pool, extracted in aqueous buffer, and the solids removed by centrifugation. Total protein determined was by a commercial Coomassie dye binding assay (Bio-Rad) or BCA assay (Pierce Chems.) with bovine IgG as a standard. Extracts were screened by the ELISA and Western methods as above. As shown in FIG. 14, a number of independent events were identified with expression greater than 1% of total seed protein. Some of these events are represented by multiple ears, with each showing similar expression levels. The ratio of positive seed to negative seed expression was generally as expected for each event: for selfed ears, a 3:1 ratio is expected, and for outcrossed, a 1:1 ratio is expected. When second generation seed was tested, even higher expression was noted, presumably due to higher gene dose. Reduced SDS-PAGE Western blot indicated significant material of the correct mobility was seen in seed of multiple first generation events, though a truncation product was also observed. FIG. 15.

Partial hGH Purification, N-terminal Amino Acid Sequencing, and Quality Tests from Corn

Seeds from multiple first generation transgenic events were pooled, ground to a fine powder, and the hGH purified. The powder was mixed with ten volumes of 100 mM Tris buffer, and shaken for one hr at room temperature. The material was centrifuged, the top fatty layer removed, and the remainder poured through cheesecloth to recover 163 ml of fluid.

The material was loaded at 2 ml/min. onto a Gibco Q HB2 column (10×75 mm) (Life Technologies, Rockville, Md.), equilibrated in 25 mM Tris, 10 mM NaCl, pH 8.3, washed with ten volumes of equilibration buffer, and developed with 1 M NaCl. Fractions of 1.5 ml were collected. The flow through was reloaded on the column, rewashed, and developed with a step change to 1M NaCl at 0.8 ml/min flow rate, with 1.6 ml fractions collected. The fractions with the highest hGH levels from the two runs were pooled, and concentrated with buffer exchange to 20 mM Tris pH 9 using an Amicon YM30 membrane (Millipore, Bedford, Mass.). This was loaded to a 5 ml BioRad High Q column (Bio-Rad Labs.), equilibrated in 25 mM Tris, 10 mM NaCl, pH 9. It was developed with a linear gradient to 1 M NaCl, with 5 ml fractions taken. Comparision of hGH levels by ELISA to total protein levels indicated a purity of 1.1% at 225 mg/L.

The major fractions were subjected to amino terminal sequencing as follows. The major fractions were applied to a reducing 4-20% gradient SDS-PAGE, and then the SDS/PAGE-separated proteins were transferred onto a polyvinylidene difluoride (PVDF) membrane (Schleicher & Schuell, Inc., Keene, N.H.). The blots were stained with 0.1% Ponceau S (Sigma, St. Louis, Mo.) in 1% acetic acid, then destained in water. The upper band corresponding to the appropriate size for hGH as seen in the Western blot above was marked and then sequenced on an Applied Biosystems sequencer (Applied Biosystems, Foster City, Calif.). The sequencing results revealed the preferred result of only the nature-identical N-terminus, PheProThr, being present without the presence of any hydroxyproline.. Additional sequencing gave the sequence SerHisAsn. This would be consistent with hydrolysis before ser150in hGH. Under reduced conditions, the AA1-149 fragment is observed on the Western blot above.

To determine in vitro hGH activity, a cell proliferation-based test similar to the method of Dattani et al. was performed. Dattani et al., 270 J. Biol Chem. 9222-26 (1995). Mammalian rat lymphoma Nb2 cells that respond to hGH were incubated with varying levels of samples, and cell proliferation determined by the proportional conversion of tetrazolium dye to a colored formazan product (Promega). The cells exhibited a positive, dose-dependent stimulation. More specifically, FIG. 16 shows the partially purified corn sample has a similar specific activity as the standard material spiked into null corn extract at a similar dilution. Activity tests compared the corn material to E. coli-produced hGH spiked into non-producing corn seed extract processed in a similar way, at 0.001 to 10 ng/ml hGH levels. A control null corn seed extract was used at similar dilutions. The corn-produced and the E. coli-produced hGH showed bioactivity.

Mass Spectrometry of Phe-hGH

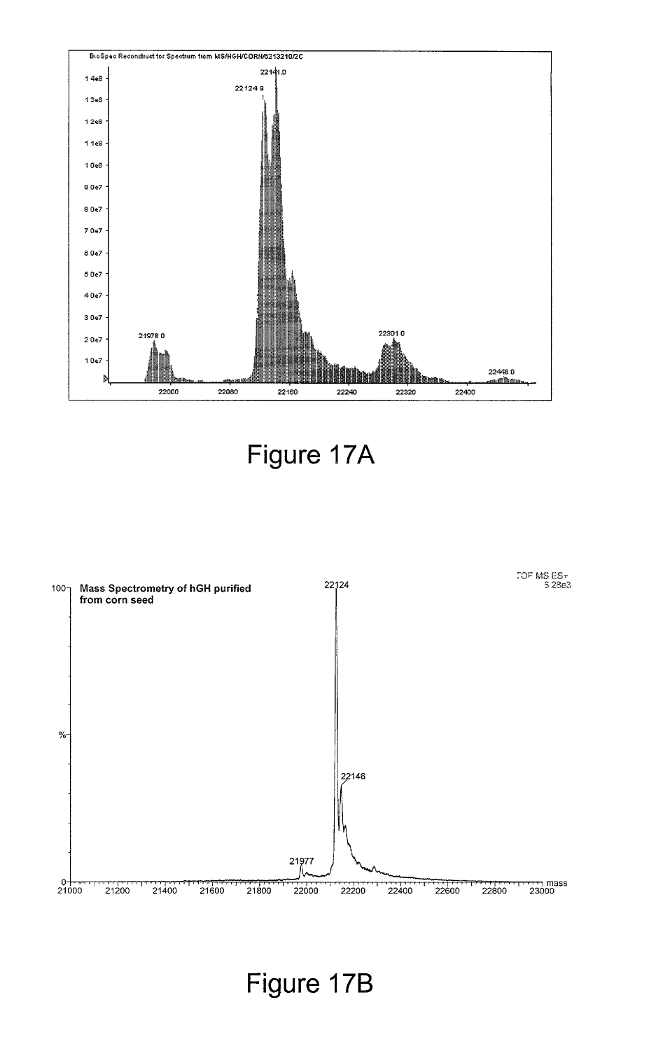

Following further purification by reverse phase HPLC, the major fractions of Phe-hGH were also analyzed by mass spectrometry. Mass spectrometry indicated recovery of significant levels of authentic-sized hGH at 22,125 daltons that had the proper disulfide linkages and was free of novel glycosylation and amino acid modification. FIG. 17A. A later major peak at 22141 mass units is most likely related to the hydrolyzed but nonreduced hGH, which yielded the sequence breakpoint around Ser150 as described above.

Large Scale Purification of hGH from Corn Seed

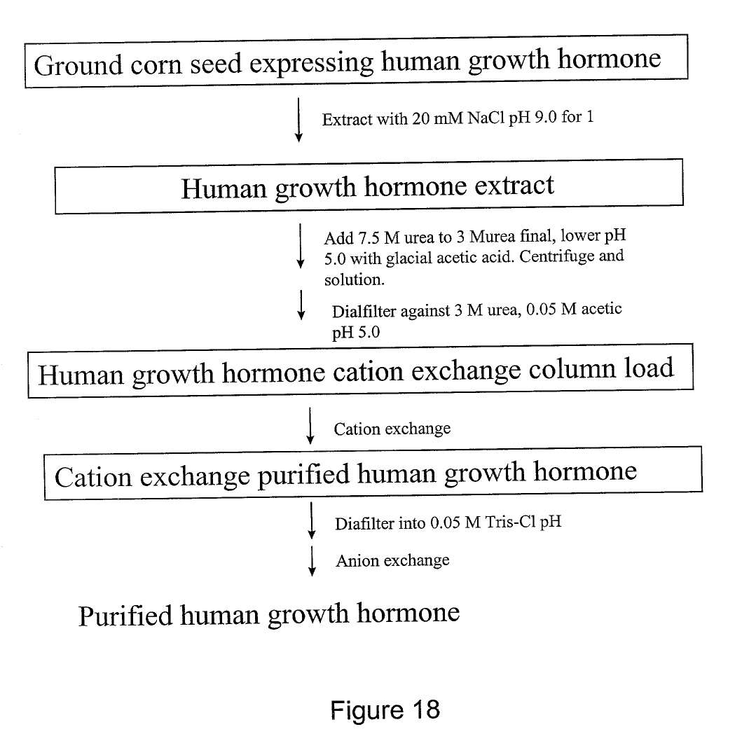

One hundred grams of ground corn seed was added to 1000 mls of 20 mM NaCl. While stirring, the pH of the solution was raised to 9.0+/−0.1 with 2.5 M NaOH. See FIG. 18. The extract was stirred for one hour at room temperature. After one hour, the extract was filtered through MIRACLOTH™ (Novagen, Madison, Wis.). Deionized urea (7.5 M) was added to the filtered material to a final urea concentration of 2.9-3.1 M. The pH of the solution was lowered to 5.0+/−0.1 with glacial acetic acid over a period of twenty minutes at room temperature. The solution was then centrifuged at 10,000 rpm in a Sorvall™ GSA rotor (Kendro Lab. Prods., Newtown, Conn.) for thirty minutes. The supernatant was decanted and filtered through a 0.45 micron filter. The supernatant was diafiltered against ten turnover volumes (TOVs) with a 10,000 dalton cutoff (Millipore™, Bedford, Mass.) tangential flow cartridge. The diafiltration buffer was 3 M urea, 0.05 M acetic acid, pH 5.0.

The sample was loaded onto a CM-SEPHAROSE™ (2.2×20 cm) column (Amersham, Piscataway, N.J.) equilibrated with 3 M urea, 0.05 M acetic acid, pH 5.0 at a flow rate of four column volumes/hour (CVs/hour). After loading, the column was washed with four CVs of 3 M urea, 0.05 M acetic acid pH 5.0. Bound hGH was eluted with a 54 CV linear gradient of 0-0.20 M NaCl in 3 M urea 0.05 M acetic acid pH 5.0 was done. Fractions were collected every 0.30 CVs. Fractions were analyzed by RP-HPLC, BCA protein assay, and cation exchange HPLC. Fractions containing greater than 40% hGH (by RP-HPLC)/mg/ml total protein (by BCA) were pooled for anion exchange chromatography. Four 100 gram corn seed extractions were purified through cation exchange chromatography.

The four cation exchange pools were combined, concentrated and diafiltered against ten TOVs of 0.05 M Tris-Cl, pH 7.5 with a 10,000 dalton cutoff MILLIPORE® tangential flow cartridge. The diafiltered pool was loaded onto a 1.6 by 20 cm Q-SEPHAROSE™ (Pharmacia Amersham, Piscataway, N.J.) equilibrated with 0.05 M Tris-Cl, pH 7.5. The flow rate was 4.5 CVs/hour. After loading, the column was washed with one CV of 0.05 M Tris-Cl, pH 7.5. A 30 CV linear gradient of 0-0.15 M NaCl in 0.05 M Tris-Cl pH 7.5 was run. Fractions were collected every 0.2 CVs. Fractions were analyzed by RP-HPLC, absorbance at 280 run and anion exchange HPLC. Fractions containing greater than 98% hGH based on anion exchange HPLC were pooled.

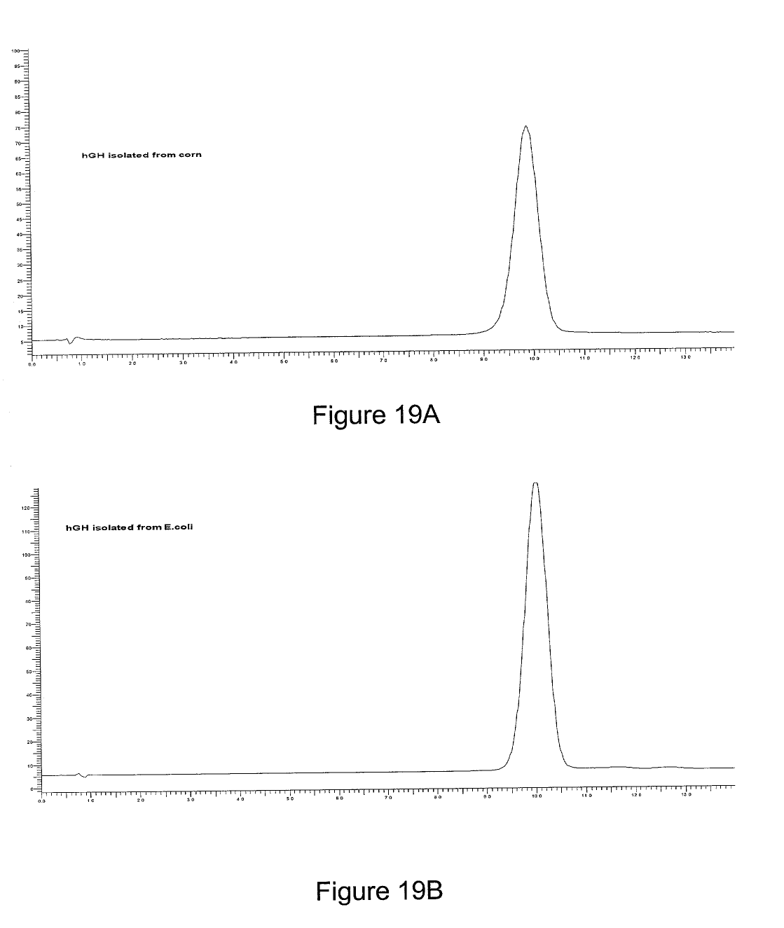

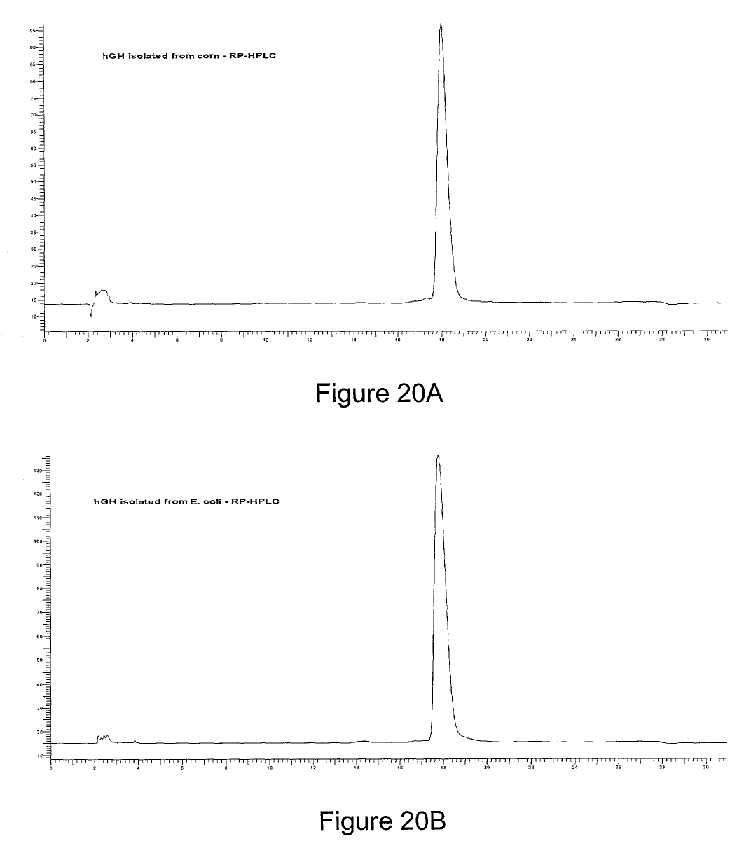

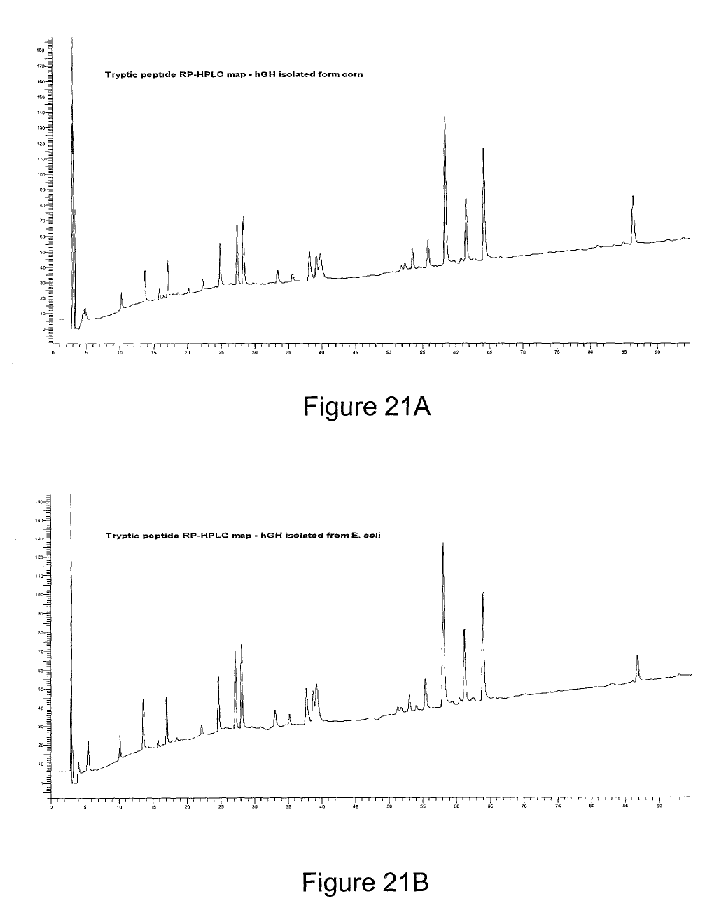

The hGH recovered from the anion exchange pool was compared to hGH molecule purified from recombinant E. coli by anion exchange HPLC (FIGS. 19A-B), RP-HPLC (FIGS. 20A-B), mass spectrometry (FIGS. 17A-B) and tryptic peptide mapping (FIGS. 21A-B). All three assays showed similar HPLC profiles for the hGH purified from corn compared to hGH purified from E. coli. Amino terminal sequencing and electrospray mass spectrometry of hGH isolated from corn seed showed that an intact hGH molecule with the correct amino terminus had been produced in corn without hydroxyproline or sugar additions. The purification steps in this Example also removed the cleaved form of hGH. Sequencing of an earlier fraction from this purification scheme had showed cleavage near amino acid residue Ser150.

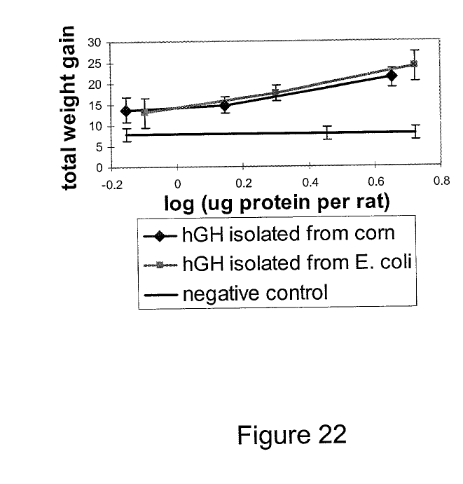

A bioassay compared the hGH obtained from this large-scale corn purification to that purified from E. coli. Rats were treated with hGH as described in 23 Pharmacopedeial Forum 4671 (1997), and their weight gain was compared to the non-treated control rats. The data shown in FIG. 22 indicates that the corn-produced hGH has a similar dose response compared to the E. coli-produced material.

Finally, regarding purification, cation exchange chromatography can greatly facilitate the initial purification of transgenic proteins from plants that have an acidic pI. Most transgenic proteins will bind to the cation resin, but most corn proteins will not.

### Example 8

Transient Expression of G-CSF with Different Targeting Signals

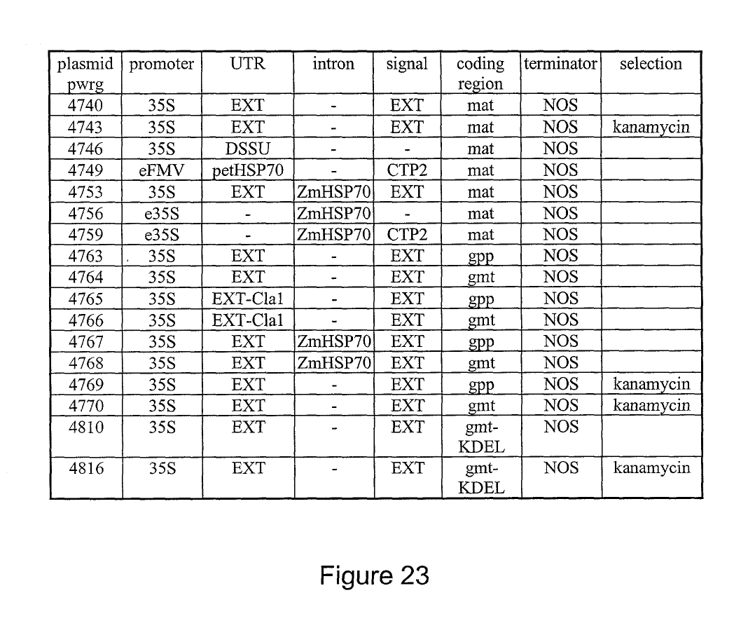

A plasmid containing the G-CSF coding region, that was originally designed for expression in E. coli, was recloned into a plant expression vector. In the E. coli expression vector, the G-CSF gene had been preceded immediately by methionine and alanine codons for the direct expression of the protein, in the context of a NcoI restriction enzyme site, directly before the nature-identical G-CSF ThrProLeu N-terminus. This G-CSF coding sequence had been further modified by performing a cys17ser change (Kuga et al., 159 Biochem. Biophys. Res Comm. 103-111 (1989)), to minimize the potential of incorrect disulfide linkages during E. coli expression and refolding. The entire set of G-CSF vectors is in FIG. 23.

Three expression vectors were constructed that resulted in three different forms of G-CSF. These expression vectors consisted of a cytosolic form, a secreted form, and a plastid form. The first expression vector for the cytosolic form included the G-CSF gene, the CaMV 35S promoter, a plant active 5′UTR, and a 3′UTR/Nos poly A signal. The cytosolic expression vector yielded MetAlaThr as a translation start site. The expression vector for the secreted form contained the G-CSF structural gene, a 5′UTR that also contained a signal peptide to facilitate secretion of the nascent protein through the endoplasmic reticulum, and a 3′UTR/Nos poly A signal. This expression cassettes comprised a AlaSerAla/MetAlaThr (SEQ ID NO:13) fusion point between the signal peptide and the N-terminus, which will lead to a methionine N-terminus during secretion. Finally, the third expression vector, which is the plastid form, comprised the G-CSF expression cassette fused to the CaMV 35S promoter, a 5′ UTR that also contained a plastid targeting sequence, and a 3′UTR/Nos poly A addition signal. Also, an intron from the corn heat shock 70 gene was placed in between the promoter and signal peptide. This expression vector was designed to yield a CysMetLeuAla/MetAlaThr (SEQ ID NO:14) fusion point, that is expected to generate a methionine N-terminus on the expressed G-CSF protein after import to the plastid. Expression vectors without the intron were the same, except that the plastid version used an FMV promoter.

The expression vectors were delivered into soy hypocotyls and corn leaves by particle bombardment as described above. Following delivery, transgenic plants were analyzed for the expression of the three forms of G-CSF via Western blotting with a rabbit-anti-G-CSF specific antibody. Total soluble protein was extracted from about 250 mg of tissue of transgenic tisue in 0.5 ml of extraction buffer (25 mM Tris-acetate (pH 8.5), 0.5 M NaCl, 5 mM PMSF). The homogenate was centrifuged at 12,000 ×g for 10 minutes. Protein concentration in the supernatant was measured by a Bradford assay. Proteins were separated by reducing SDS/PAGE (4-20%).

For Western blotting, the SDS/PAGE-separated proteins were transferred onto a nitrocellulose membrane (Amersham). The blots were probed with a rabbit-anti-G-CSF antibody, and detected with goat-anti-rabbit Ig-conjugated horse radish peroxidase, followed by ECL reagent (Amersham).

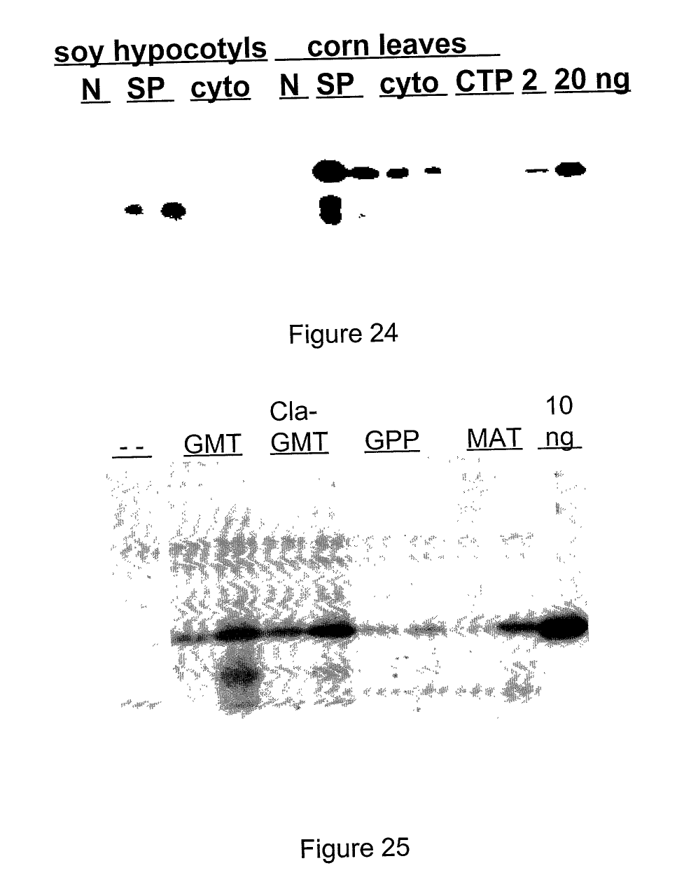

The results show that the plant hosts could support the production of G-CSF. FIG. 24. Truncation products are more prevalent with soy than corn, and more signal of the proper size is seen with corn. Expression in both systems was greater with a secretion signal (SP) than with a cytosolic signal. Expression was not detected with the plastid signal (CTP2).

Expression of G-CSF with Different Codon Usage

Since the expression vector containing the secretion signal peptide provided the best expression results in the plant host, the vector was modified to alter the G-CSF N-terminus fusion to the signal peptide, incorporate the natural ser17, and alter codon usage to improve expression levels as described previously. The modified vectors yielded a fusion point between the signal peptide and the G-CSF N-terminus of AlaSerAla/MetThrProLeu (SEQ ID NO:07) met-G-CSF), expected to yield a G-CSF amino acid sequence with a methionine terminus and cys17, identical to commercial NEUPOGEN® (Amgen). These vectors were delivered into corn leaves and analyzed as described above. The results in FIG. 25 shows accumulation from several different vectors with modified codons (mat, gmt, gpp, nsi), similar to that seen with the earlier secreted codon design in terms of relative presence of full sized compared to truncated product.

Expression of G-CSFwith a Carboxy-Terminal Fusion

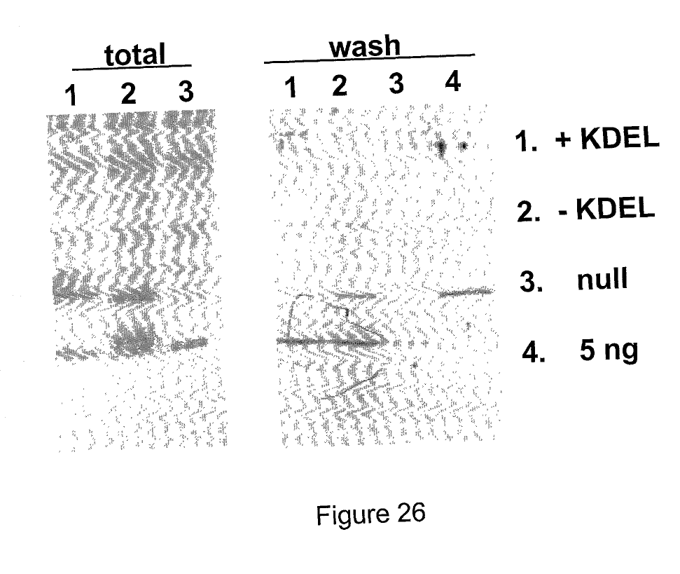

A carboxy-terninal “KDEL” fusion was added to the secreted G-CSF expression vector, yielding a carboxy-terminal fusion point of AlaGlnPro/AspAspLysGluAspLeu (SEQ ID NO:08). This design has been used to increase expression of other proteins, presumably by stopping the secretion of the protein before traversing the golgi and later secretory compartments. The newly modified expression vector was named pwrg4810. The pwrg4810 expression vector was delivered into corn leaves, extracted for total proteins, separated by reducing SDS-PAGE, and analyzed by Western blot for G-CSF as above. To determine if the KDEL (SEQ ID NO:09) sequence influences secretion of the attached G-CSF, additional plant tissue after harvest also was submerged in PBS for 30 min on ice, and the PBS collected, and analyzed by Western blot. The Western blot of FIG. 26 shows most lanes have a low mobility contaminating signal. Comparing lanes 1 and 2 (“total” blot) indicates the KDEL fusion from total leaf extracts has an expected slower mobility relative to the secreted version (total lane 1 compared with lane 2). The KDEL fusion also leads to less truncation product than the secreted form. When the cell washes were analyzed, signal with G-CSF mobility is only seen with the secreted version (wash lane 2 compared with lane 1). This indicates the signal peptide fusion to GCSF allowed secretion, and subsequent truncation product accumulation, but the KDEL fusion arrested secretion, and improved yield quality for this class of molecule.

### Example 9

Stable Tobacco Cell Expression of G-CSF with Different Targeting Signals

Some of the G-CSF expression vectors described in Example 8 were used to generate stable transgenic tobacco cell culture. These expression vectors included the cytosolic form, the secreted form, the plastid form, and the KDEL fusion. The secreted forms included designs with different codon usage. These expression cassettes were mixed with a kanamycin resistance cassette, or the two cassettes were developed into a single vector, and then co-transformed by accelerated particle delivery as in Example 4.

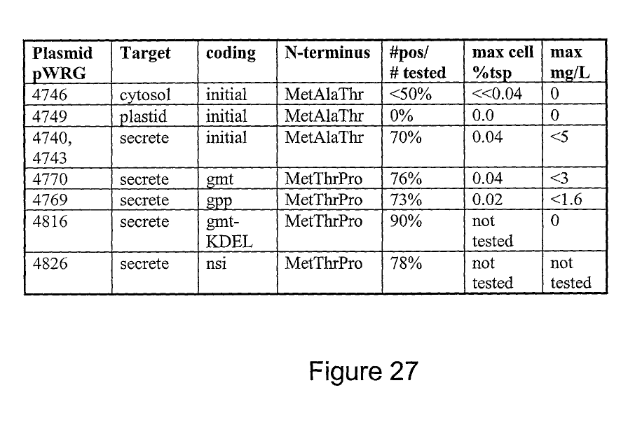

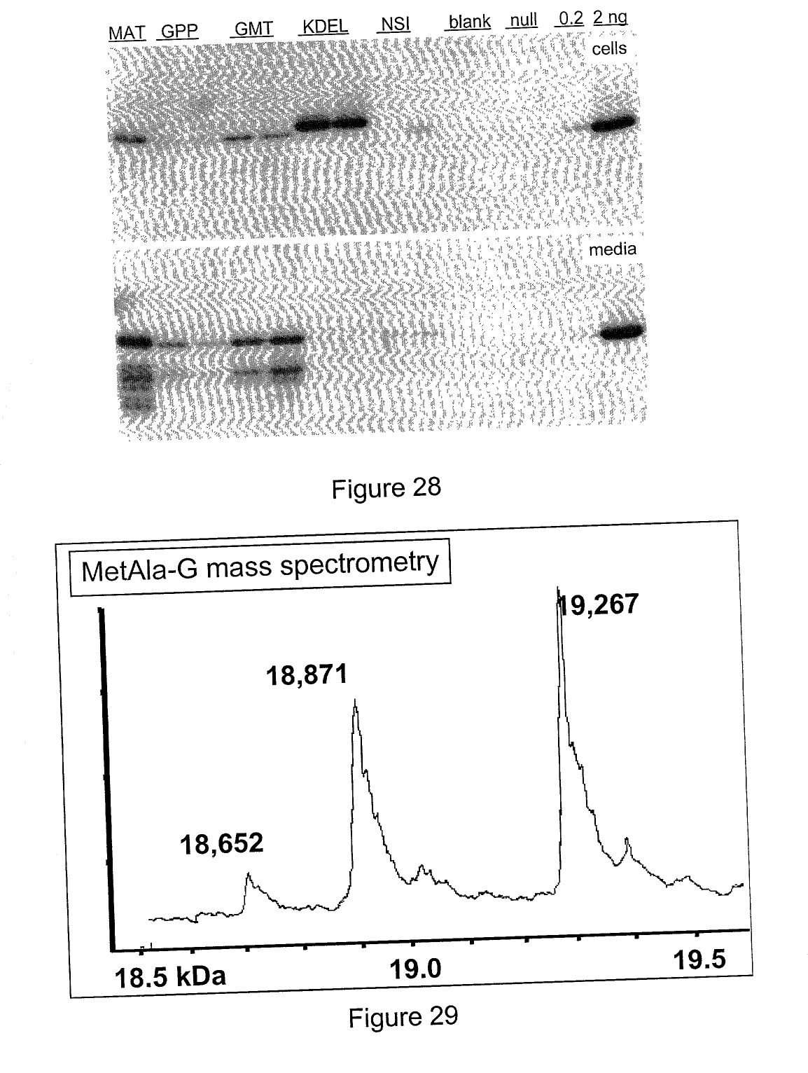

Expression of G-CSF in transgenic suspension cells was evaluated by ELISA. The appropriate colonies were then advanced to liquid suspension culture and re-tested for G-CSF accumulation in the media and cell extracts, as summarized in FIG. 27. The ELISA results indicated detectable signal from all vector designs, except with plastid targeting. Reduced SDS-PAGE Western blots of 18.5 1g total cell extract protein was compared to 10 μl suspension media from the same lines. FIG. 28. The major band detected showed the expected mobility: similar to the G-CSF standard, except slightly slower mobility for the KDEL fusion. When the media was examined, no signal was seen for the KDEL design, presumably because the protein is retained within the secretory path. The secreted forms also had significant truncation bands. The KDEL may then be valuable if the attached protein was purified from the whole cells. Designs which would allow later accurate removal of the KDEL, or allow retention in the secretory path without a fusion, may help minimize degradation, while still making the desired protein sequence.

Plant Cell MetAla-G-CSF Purification and Quality Tests

MetAla-G-CSF was purified from the media of the tobacco cell line transformed with the secreted expression vector pwrg4743, having the AlaSerAla/MetAlaPhe (SEQ ID NO:03) fusion between the signal peptide and the N-terminus of G-CSF. Media was collected four days post-inoculation, the pH adjusted to pH 3.6 with HCl, then loaded on a SBB cation exchange column (Amersham, Piscataway, N.J.). The column was washed with 10 mM NaAc pH 4, and then the G-CSF was eluted with a linear salt gradient at pH 4,250 mM NaCl. The major fractions were pooled and applied to a POROS HS cation exchange (Amersham, Piscataway, N.J.). Next, the column was washed in 50 mM NaCitrate pH 3.6, and then developed with a pH 3.6 to 7.5 gradient. G-CSF was eluted at pH6.3. This pool was applied to a Macroprep-Q column (Amersham, Piscataway, N.J.), washed with 25 mM Tris-Cl pH 9.2, and developed with a 0 to 200 mM NaCl gradient. G-CSF eluted at 75 mM NaCl, pH 9.2. The final material was 98% pure, determined by comparing G-CSF ELISA signal to total protein using a Coomassie Plus assay and bovine IgG as a standard(Pierce Chemicals, Rockford, Ill.). Comparing ELISA signal of the initial to final sample showed that the process yield was 43%.

The purified material was subjected to amino terminal sequencing as follows. The final G-CSF material was applied to a reducing 4-20% gradient SDS-PAGE, and then the SDS/PAGE-separated proteins were transferred onto a PVDF membrane (Schleicher & Schuell). The blots were stained with 0.1% Ponceau S (Sigma) in 1% acetic acid and destained in water. The band corresponding to the appropriate size for G-CSF was marked and then sequenced on an Applied Biosystems sequencer. The sequencing results showed that the construct encoding a fusion of AlaSerAla/MetAlaThrProLeu (SEQ ID NO:07) generated an N-terminus amino acid sequence of MetAlaThrHypLeuGlyProAlaSerSerLeuProGln (SEQ ID NO:10). Although the signal sequence was cleaved accurately, one of the three prolines found in the sequence was modified to hydroxyproline (Hyp). Hydroxyproline is an amino acid modification, commonly seen in some secretory proteins localized to the plant cell wall.

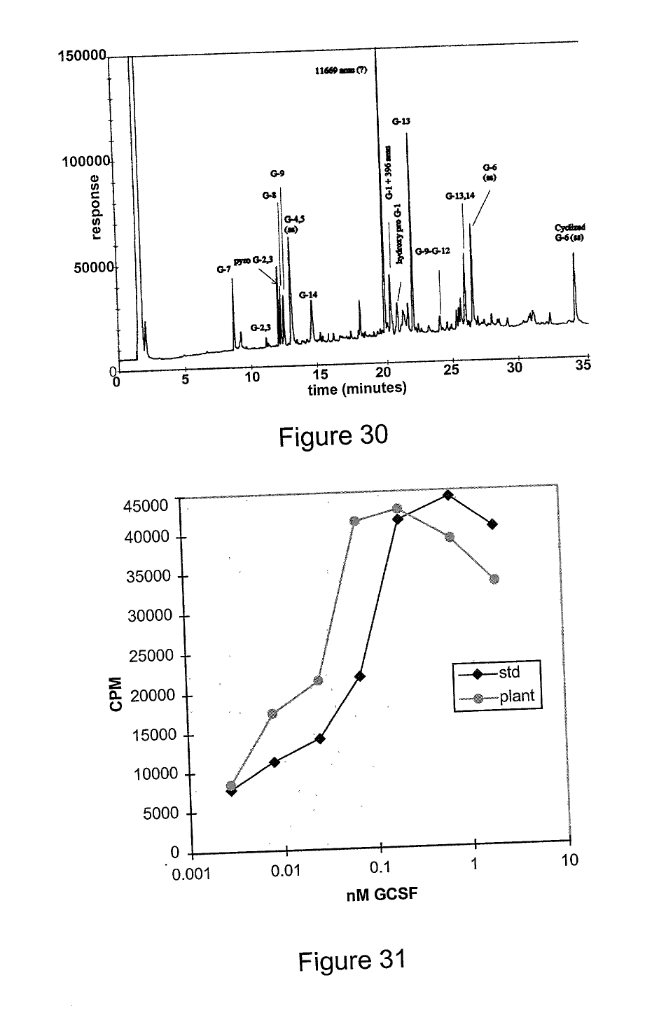

Following amino terminal sequencing, the purified G-CSF material was also analyzed by electron spray mass spectrometry (ESMS). The mass spectrometry results are shown in FIG. 29. The mass spectrometry results showed that roughly half of the purified material exhibited a molecular weight of 18,871 mass units, which is expected based upon the amino acid sequence of G-CSF. The mass spectrometry results of the remaining half of the purified material was consistent with the hydroxylation also being the site of glycosylation, which added a molecular weight of 396 mass units. Other minor peaks were interpreted as methionine oxidations, occuring either during plant accumulation, or purification. Additional mass spectrometry indicated a ladder of masses consistent with a chain of three repeating units. Similar saccharide chains of arabinose (132 mass units when polymerized) are seen in cell wall proteins. Following analysis by mass spectrometry, the purified material was also subjected to partial V-8 protease digestion followed by liquid chromatography-electron spray-mass spectrometry (LC-ESMS). The results of the peptide-mass spectrometry are shown in FIG. 30, which mapped the site of modification to the amino terminal peptide fragment of G-CSF, indicated by the peaks at 21 and 22 minutes. Moreover, the results of the peptide-mass spectrometry also indicated no evidence of O-linked glycosylation at the Thr133 position, which is generally seen in G-CSF when secreted by mammalian cells. This indicates that plants can make some amount of a non-glycosylated bioactive molecule, similar to that seen from E. coli, but without the need for refolding.

Next, a cell-based proliferation assay was performed on the purified material derived from the cells expressing secreted MetAla G-CSF. Final purified plant sample and E. coli refolded standard were each diluted to 30 μg/ml in 40 mM HEPES pH 6.3. They were used in an activity assay based on the ability of G-CSF to stimulate cell growth, as measured by ³H-thymidine uptake for incorporation into cellular DNA. The cell line used was a murine BAF3 line, transfected with the G-CSF receptor. Dong et al., 13 Mol. Cell Bio. 7774-81 (1993). The results of the proliferation assay showed positive dose-dependent activity of plant-derived G-CSF, similar to that induced by of an E. coli-derived G-CSF. FIG. 31. It is also important to note that the E. coli-derived G-CSF required ex vivo refolding, while the plant-derived G-CSF that was column purified had been properly folded in vivo.

G-CSF from Cells Transformed with Met G-CSF

G-CSF was purified from the media of the tobacco cell line transformed with the secreted expression vector pwrg4770, which contained the AlaSerAla/MetThrProLeu (SEQ ID NO:07) fusion between the signal peptide and the N-terminus of G-CSF. The column purification was performed as described above.

Following column purification, the purified material was subjected to amino terminal sequencing. The column purified G-CSF material was applied to a reducing 4-20% gradient SDS-PAGE, and then the SDS/PAGE-separated proteins were transferred onto a PVDF membrane (Schleicher & Schuell). The blots were stained with 0.1% Ponceau S (Sigma) in 1% acetic acid and destained in water. The band corresponding to the appropriate size for G-CSF was marked and then sequenced on an Applied Biosystems sequencer. The sequencing results showed the presence of the MetThrHypLeu N-tenninus, rather than the desired MetThrProLeu (SEQ ID NO:11). Mass spectrophotometry indicated the sample was 18814 mass units, compared to the predicted 18815 mass for full length G-CSF, 2 disulfide bonds, and one hydroxyproline. This indicates that while the plant-modified amino acid hydroxyproline was present, sugars were not added. This is different than the results seen with the MetAla design of G-CSF.

All references, patents, or applications cited herein are incorporated herein by reference in their entirety, as if written herein.

We claim:

1. A method for producing a cytokine in a plant host system wherein said plant host system has been transformed with a chimeric nucleic acid sequence encoding said cytokine, comprising the step of:
cultivating said transformed plant host system under the appropriate conditions to result in the expression of said cytokine in said plant host system
wherein said cytokine accumulates to a level greater than 1% of the total soluble protein in a sample of said plant host system.

2. The method of claim 1, further comprising the step of purifying said expressed cytokine from said plant host system.

3. The method of claim 1, wherein said expressed cytokine is free from amino acid modifications.

4. The method of claim 3, wherein said amino acid modification comprises the addition of hydroxyproline to said cytokine.

5. The method of claim 1, wherein said cytokine is free of novel glycosylation.

6. The method of claim 1, wherein said chimeric nucleic acid sequence comprising:
a first nucleic acid sequence capable of regulating the transcription in said plant host system of a second nucleic acid sequence wherein said second nucleic acid sequence encodes a signal sequence is linked in reading frame to a third nucleic acid sequence encoding a cytokine.

7. The method of claim 6, wherein said nucleic acid sequence further comprises a fourth nucleic acid sequence linked in reading frame to the 3′ end of said third nucleic acid sequence.

8. The method of claim 7, wherein said fourth nucleic acid sequence encodes a “KDEL” amino acid sequence.

9. The method of claim 6, wherein said nucleic acid sequence capable of regulating transcription comprises a plant active promoter.

10. The method of claim 6, wherein said second nucleic acid sequence is capable of targeting said cytokine to a sub-cellular location within a plant host system.

11. The method of claim 10, wherein said sub-cellular location comprises the cytosol.

12. The method of claim 10, wherein said sub-cellular location comprises a plastid.

13. The method of claim 10, wherein said sub-cellular location comprises the endoplasmic reticulum.

14. The method of claim 6, wherein said second nucleic acid sequence comprises a sufficient portion of ubiquitin.

15. The method of claim 14, wherein said ubiquitin comprises an ubiquitin monomer derived from yeast.

16 The method of claim 15, wherein said ubiquitin comprises an ubiquitin monomer of potato ubiquitin gene 3.

17. The method of claim 6, wherein said second nucleic acid sequence comprises a sufficient portion of an oleosin protein to provide targeting within said plant host system.

18. The method of claim 17, wherein a nucleic acid sequence encoding an amino acid sequence that is specifically cleavable by enzymatic or chemical means is included between said second nucleic acid sequence encoding said oleosin protein and the third nucleic acid sequence encoding a cytokine.

19. The method of claim 18, wherein a nucleic acid encoding said oleosin protein is derived from soy.

20. The method of claim 1, wherein said cytokine is a member of the cytokine superfamily selected from the group consisting of TGF-beta, PDGF, EGF, VEGF; chemokines; and FGFs.

21. The method of claim 20, wherein said cytokine comprises hGH.

22. The method of claim 20, wherein said cytokine comprises G-CSF.

23. A plant host system that has been transformed with a chimeric nucleic acid sequence wherein said chimeric nucleic acid sequence comprises:
a first nucleic acid sequence capable of regulating the transcription in said plant host system of a second nucleic acid sequence wherein said second nucleic acid sequence encodes a signal sequence that is linked in reading frame to a third nucleic acid sequence encoding a cytokine.

24. The method of claim 23, wherein said nucleic acid sequence further comprises a fourth nucleic acid sequence linked in reading frame to the 3′ end of said third nucleic acid sequence.

25. The method of claim 24, wherein said fourth nucleic acid sequence encodes a “KDEL” amino acid sequence.

26. The plant host system of claim 23, wherein said first nucleic acid sequence comprises a plant active promoter.

27. The plant host system of claim 23, wherein said signal sequence capable of targeting said cytokine to a sub-cellular location within said plant host system.

28. The plant host system of claim 23, wherein said signal sequence is capable of targeting said cytokine to the cytosol of said plant host system.

29. The plant host system of claim 23, wherein signal sequence is capable of targeting said cytokine to a plastid within said plant host system.

30. The plant host system of claim 23, wherein said signal is capable of targeting said cytokine to the endoplasmic reticulum located within said plant host system.

31. The plant host system of claim 23, wherein said signal sequence comprises ubiquitin.

32. The method of claim 31, wherein said ubiquitin comprises an ubiquitin monomer derived from yeast.

33. The method of claim 31, wherein said ubiquitin comprises an ubiquitin monomer of potato ubiquitin gene 3.

34. The plant host system of claim 23, wherein said signal sequence comprises a sufficient portion of oleosin to target said cytokine within said plant host system.

35. The plant host system of claim 34, wherein a nucleic acid encoding said oleosin is derived from soy.

36. The plant host system of claim 23, wherein a nucleic acid sequence encoding an amino acid sequence that is specifically cleavable by enzymatic or chemical means is included between said signal sequence and said third nucleic acid sequence encoding a cytokine.

37. The plant host system of claim 36, wherein said cleavable amino acid sequence comprises enterokinase.

38. The plant host system of claim 36, wherein said signal sequence comprises a sufficient portion of oleosin protein to target said cytokine within said plant host system.

39. The plant host system of claim 38, wherein a nucleic acid sequence encoding said oleosin protein is derived from soy.

40. The plant host system of claim 23, wherein cultivating said plant host system under the appropriate conditions results in the expression of said cytokine.

41. The plant host system of claim 40, wherein said expressed cytokine is purified from said plant host system.

42. The plant host system of claim 40, wherein said expressed cytokine is free from amino acid modifications.

43. The plant host system of claim 42, wherein said amino acid modification comprises the addition of hydroxyproline to said cytokine.

44. The plant host system of claim 40, wherein said expressed cytokine is free from novel glycosylation.

45. The plant host system of claim 23, wherein said expressed cytokine is a member of the cytokine superfamily selected from the group consisting of TGF-beta, PDGF, EGF, VEGF; chemokines; and FGFs.

46. The plant host system of claim 45, wherein said expressed cytokine comprises hGH.

47. The plant host system of claim 46, wherein the N-terminus of said expressed hGH is identical to authentic N-terminus of hGH.

48. The plant host system of claim 45, wherein said expressed cytokine comprises G-CSF.

49. The plant host system of claim 48, wherein the N-terminus of said expressed G-CSF is met-G-CSF.

50. The plant host system of claim 41, wherein said expressed cytokine is free from novel glycosylation.

51. A chimeric nucleic acid sequence capable of being expressed in a plant host system comprising:
a first nucleic acid sequence capable of regulating the transcription in said plant host system of a second nucleic acid sequence wherein said second nucleic acid sequence encodes a signal sequence is linked in reading frame to a third nucleic acid sequence encoding a cytokine.

52. The method of claim 51, wherein said nucleic acid sequence further comprises a fourth nucleic acid sequence linked in reading frame to the 3′ end of said third nucleic acid sequence.

53. The method of claim 52, wherein said fourth nucleic acid sequence encodes a “KDEL” amino acid sequence.

54. The chimeric nucleic acid sequence of claim 51, wherein said first nucleic acid sequence comprises a plant active promoter.

55. The chimeric nucleic acid sequence of claim 51, wherein said signal sequence capable of targeting said cytokine to a sub-cellular location within said plant host system.

56. The chimeric nucleic acid sequence of claim 51, wherein said signal sequence is capable of targeting said cytokine to the cytosol of said plant host system.

57. The chimeric nucleic acid sequence of claim 51, wherein signal sequence is capable of targeting said cytokine to a plastid within said plant host system.

58. The chimeric nucleic acid sequence of claim 51, wherein said signal sequence is capable of targeting said cytokine to the endoplasmic reticulum located within said plant host system.

59. The chimeric nucleic acid sequence of claim 51, wherein said signal sequence comprises ubiquitin.

60. The method of claim 59, wherein said ubiquitin comprises an ubiquitin monomer derived from yeast.

61. The method of claim 59, wherein said ubiquitin comprises an ubiquitin monomer of potato ubiquitin gene 3.

62. The chimeric nucleic acid sequence of claim 51, wherein said signal sequence comprises a sufficient portion of oleosin to target said cytokine within said plant host system.

63. The chimeric nucleic acid sequence of claim 62, wherein a nucleic acid sequence encoding said oleosin is derived from soy.

64. The chimeric nucleic acid sequence of claim 51, wherein a nucleic acid sequence encoding an amino acid sequence that is specifically cleavable by enzymatic or chemical means is included between said signal sequence and said third nucleic acid sequence encoding a cytokine.

65. The chimeric nucleic acid sequence of claim 64, wherein said cleavable amino acid sequence comprises enterokinase.

66. The chimeric nucleic acid sequence of claim 64, wherein said signal sequence comprises a sufficient portion of oleosin protein to target said cytokine within said plant host system.

67. The chimeric nucleic acid sequence of claim 66, wherein a nucleic acid encoding said oleosin protein is derived from soy.

68. The chimeric nucleic acid sequence of claim 51, wherein cultivating said plant host system under the appropriate conditions results in the expression of said cytokine.

69. The chimeric nucleic acid sequence of claim 68, wherein said expressed cytokine is purified from said plant host system.

70. The chimeric nucleic acid sequence of claim 68, wherein said expressed cytokine is free from amino acid modifications.

71. The chimeric nucleic acid sequence of claim 70, wherein said amino acid modification comprises the addition of hydroxyproline to said cytokine.

72. The chimeric nucleic acid sequence of claim 68, wherein said expressed cytokine is a member of the cytokine superfamily selected from the group consisting of TGF-beta, PDGF, EGF, VEGF; chemokines; and FGFs.

73. The chimeric nucleic acid sequence of claim 72, wherein said expressed cytokine is hGH.

74. The chimeric nucleic acid sequence of claim 73, wherein the N-terminus of said expressed hGH is identical to the authentic N-terminus of hGH.

75. The chimeric nucleic acid sequence of claim 72, wherein said expressed cytokine comprises G-CSF.

76. The chimeric nucleic acid sequence of claim 75, wherein the N-terminus of said expressed G-CSF is met-G-CSF.

77. The chimeric nucleic acid sequence of claim 68, wherein said expressed cytokine is free from novel glycosylation.

78. An expression cassette comprising a chimeric nucleic acid sequence according to claim 51.

79. A plant transformed with a chimeric nucleic acid sequence according to claim 51.

80. A plant cell culture transformed with a chimeric nucleic acid sequence according to claim 51.

81. A plant seed containing a chimeric nucleic acid sequence according to claim 51.

82. A method of preparing a bioactive, authentic mammalian growth hormone in corn plants comprising the steps of
(a) inserting a gene for said growth hormone into a corn plant expression vector;
(b) transforming corn plant cells with said expression vector;
(c) generating whole corn plants from said transformed corn cells;
(d) harvesting corn seed from whole corn plants; and
(e) purifying said growth hormone from corn seed.

83. The method of claim 82, wherein said mammalian growth hormone is human growth hormone.

84. The method of claim 82, wherein said growth hormone accumulates to a level greater than 1% of the total soluble protein in a plant sample.

85. The method of claim 84, wherein said growth hormone accumulates to level greater than 5% of the total soluble protein in a plant sample.

86. The method of claim 82, wherein said growth hormone is not glycosylated.

87. The method of claim 82, wherein said corn plant expression vector is pwrg4825.

88. Transformed corn plants and corn seed prepared by the method of claim 82.

89. A method of preparing bioactive, authentic human growth hormone from corn seed of claim 82, further comprising the steps of
(a) extracting powdered corn seed with buffered saline, wherein said extraction is carried out at a pH ranging from about pH 8 to about pH 10;
(b) adding urea to a concentration of about 2M to 3.5 M urea;
(c) adjusting the pH of the extract to about pH 5;
(d) clarifying the solution;
(e) purifying by cation exchange chromatography, wherein said cation exchange chromatography is carried out in the presence of urea at a pH from about 4.5 to about 5.5; and
(f) purifying by anion exchange chromatography, wherein said anion exchange chromatography is carried out in the absence of urea at a pH from about 7.0 to about 8.0.

90. A cytokine that is produced from a plant host system expressing a nucleic acid sequence wherein said nucleic acid sequence comprises:
a first nucleic acid sequence capable of regulating the transcription in said plant host system of a second nucleic acid sequence wherein said nucleic acid sequence encodes a 5′ regulatory region is linked in reading frame to a third nucleic acid sequence encoding a cytokine.

91. A method for producing a cytokine in a plant host system wherein said plant host system has been transformed with a chimeric nucleic acid sequence encoding a cytokine, comprising the step of:
cultivating said transformed plant host system under the appropriate conditions to result in expression of said cytokine, wherein said expressed cytokine is free from amino acid modifications in said plant host system.

92. A method for producing a cytokine in a plant host system wherein said plant host system has been transformed with a chimeric nucleic acid sequence encoding a cytokine, comprising the step of:
cultivating said transformed plant host system under the appropriate conditions to result in expression of said cytokine, wherein said expressed cytokine is free novel glycosylation in said plant host system.


SEQUENCE LISTING (14 sequences)

SEQ ID NO: 1 - DNA, 72 bp, Artificial Sequence
  Description of Artificial Sequence Oligomer dar 139
ttagctagcgaaagctccgccttcccgactatcccactgagccgcctgttcgacaacgctatgctgcgagct

SEQ ID NO: 2 - DNA, 65 bp, Artificial Sequence
  Description of Artificial Sequence Oligomer dar 140
cgcagcatagcgttgtcgaacaggcggctcagtgggatagtcgggaaggcggagctttcgctagc

SEQ ID NO: 3 - protein, 6 aa, Homo sapiens
ASAMAF

SEQ ID NO: 4 - protein, 7 aa, Homo sapiens
CMLAMAF

SEQ ID NO: 5 - protein, 7 aa, Homo sapiens
LRGGFPT

SEQ ID NO: 6 - protein, 8 aa, Homo sapiens
DDDDKFPT

SEQ ID NO: 7 - protein, 7 aa, Homo sapiens
ASAMTPL

SEQ ID NO: 8 - protein, 9 aa, Homo sapiens
AQPDDKEDL

SEQ ID NO: 9 - protein, 4 aa, Homo sapiens
KDEL

SEQ ID NO: 10 - protein, 13 aa, Homo sapiens
  MOD_RES (4): hydroxyproline
MATXLGPASSLPQ

SEQ ID NO: 11 - protein, 4 aa, Homo sapiens
MTPL

SEQ ID NO: 12 - protein, 191 aa, Homo sapiens
FPTIPLSRLFDNAMLRHARLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 13 - protein, 6 aa, Homo sapiens
ASAMAT

SEQ ID NO: 14 - protein, 7 aa, Homo sapiens
CMLAMAT